(19) United States
(12) Patent Application Publication
(10) Pub. No.: US 2014/0004571 A1
(43) Pub. Date: Jan. 2, 2014

(54) COMPOSITIONS AND METHODS FOR BIOMASS LIQUEFACTION

(71) Applicant: BP Corporation North America Inc., Houston, TX (US)

(72) Inventors: James B. Garrett, San Diego, CA (US); Justin T. Stege, San Diego, CA (US); William M. Lafferty, San Diego, CA (US); Arne I. Solbak, JR., San Diego, CA (US); Sarah Richardson Hanson, San Diego, CA (US); John Poland, San Diego, CA (US)

(73) Assignee: BP Corporation North America Inc., Houston, TX (US)

(21) Appl. No.: 13/692,921

(22) Filed: Dec. 3, 2012

Related U.S. Application Data
(60) Provisional application No. 61/566,275, filed on Dec. 2, 2011.

(51) Int. Cl. C12P 19/14

(52) U.S. Cl. CPC C12P 19/14
USPC 435/99

(57) ABSTRACT

The present disclosure relates methods and compositions for pretreatment of biomass, for example to form a biomass slurry suitable for downstream processing (e.g., saccharification, fermentation, etc.). The present methods provide several advantages in industrial operations, such as better heat exchange, reduced power and water usage, and the ability to carry out reactions continuously due to the reduced requirement for cleaning reaction vessels.

### 1. PRIORITY

This application claims priority under 35 U.S.C. §119(e) of provisional application No. 61/566,275 filed on Dec. 2, 2011, the contents of which are hereby incorporated by reference in their entireties.

### 2. BACKGROUND

Cellulose is an unbranched polymer of glucose linked by β(1→4)-glycosidic bonds. Cellulose chains can interact with each other via hydrogen bonding to form a crystalline solid of high mechanical strength and chemical stability. The cellulose chains are depolymerized into glucose and short oligosaccharides before organisms, such as the fermenting microbes used in ethanol production, can use them as metabolic fuel. Cellulase enzymes catalyze the hydrolysis of the cellulose (hydrolysis of β-1,4-D-glucan linkages) into products such as glucose, cellobiose, and other cellooligosaccharides.

In lignocellulosic biomass, crystalline cellulose fibrils are embedded in a less well-organized hemicellulose matrix which, in turn, is matrixed with a complex lignin structure. Naturally occurring biomass is recalcitrant to full hydrolysis by cellulases: treatment of naturally occurring cellulosic materials with cellulases generally results in cellulose hydrolysis yields that are less than 20% of theoretically predicted results. Hence, some “pretreatment” of the biomass is typically carried out prior to attempting the enzymatic hydrolysis of the cellulose and hemicellulose in the biomass. Pretreatment refers to a process that converts lignocellulosic biomass from its native form into a form that is more amenable to cellulose hydrolysis. Compared to untreated biomass, pretreated biomass is characterized by an increased surface area (porosity) accessible to cellulase enzymes, and solubilization or redistribution of lignin. Nonetheless, the pretreated biomass exhibits high viscosity when incorporated into a liquid phase for saccharification. The viscous slurry is difficult to handle, limits the concentration of biomass in reactions, and reduces the efficiency of saccharifying enzymes. Geddes et al., 2010, Bioresource Technology 101 (23):9128-9136, treated a10% slurry of phosphoric acid-treated sugar cane bagasse with cellulase for 2 or 6 hours at bench scales and at temperatures of up to 60° C. to reduce slurry viscosity, but did not achieve a slurry viscosity reduction at 70° C. or 80° C. Accordingly, there is a need in the art for liquefaction processes that reduce the viscosity of biomass slurries to improve their ease of handling, enable higher reaction concentrations, and increase saccharification efficiency under conditions suitable for industrial processes.

### 3. SUMMARY

The present disclosure relates to the pretreatment of biomass to make it more amenable for downstream processing, for example in a saccharification or fermentation process. In the present methods and compositions, biomass is formed into a slurry and treated with one or more enzymes such as cellulases to make it more suitable for handling in the downstream reactions, a process referred to herein as “liquefaction”. Without being bound by theory, the inventors believe that the liquefaction methods disclosed herein reduce viscosity of biomass slurry and/or effect depilling of cellulose fibers, even under conditions where enzymatic saccharification of cellulose is minimal. The liquefaction methods, particularly when applied to biomass that has been steam exploded, result in biomass that is suitable for downstream processes in which biomass is hydrolyzed to sugar monomers, which in turn can be transformed into other molecules, including fuel molecules such as ethanol.

The hydrolysis of biomass into fermentable sugars, a process referred to as “saccharification,” decreases in efficiency as percentages of biomass solids increase above 10% (weight percentage). This decrease is thought to be due to properties of the biomass such as the high viscosity of such slurries, which prevents efficient mixing and resulting in slower diffusion of enzymes to the substrate. When slurries are treated with hydrolyzing proteins prior to saccharification according to the methods disclosed herein, the liquefaction of the slurry allows it to be transferred (e.g., pumped) more readily from one reaction vessel to another and makes it more accessible to hydrolysis into sugar monomers in saccharification processes. Biomass liquefaction also enables the pumping and processing of slurries with significantly higher consistencies. Advantageously, liquefaction can be carried out at relatively high temperatures (greater than 60° C., preferably 62° C. or greater), which minimizes the degree of saccharification and minimizes microorganism growth in the reactor, which in turn prevents microorganisms that could compete with the fermenting microorganism from reaching the fermentation tank in an industrial process. Moreover, liquefied biomass allows better temperature control during saccharification as it is less likely to clog heat exchangers, and allows reaction vessels to operate continuously due to reduced clogging of the heat exchangers and distillation column plates, and concomittant reduced cleaning requirements. The improved mixability and flowability of a liquefied biomass also contributes to improved temperature control, and also improves pH control as well as mass transfer and enzyme rates. Further, the use of liquefied biomass reduces the need for water usage, both at the level of slurry formation and saccharification, which can occur at much higher solids content than would be permissible in the absence of liquefaction, and also during downstream processing, e.g., centrifugation or fermentation products. Accordingly, the present methods permit the use of smaller reactors, which provides capital cost savings. Additionally, the methods of the present disclosure result in rapid liquefaction. The reduction in reaction times translates to a reduction in operational costs. The liquefaction methods of the disclosure further result in higher yields and higher concentrations of fermentation products.

Accordingly, the disclosure generally provides methods for processing or processing of biomass slurry/pretreating biomass/liquefying biomass/preparing biomass for downstream processing. In certain aspects, the methods involving incubating the biomass slurry (or biomass solids and an aqueous phase, such as water and/or a hemicellulose hydrolysate, which are the components for making biomass slurry) with one or more hydrolyzing proteins, wherein:
(a) the hydrolyzing proteins (i) are characterized by a cellulase activity of 10 CTU to 500 CTU cellulase per gram dry weight of solids in the slurry (e.g., 10 CTU, 20 CTU, 40 CTU, 60 CTU, 80 CTU, 100 CTU, 125 CTU, 150 CTU, 175 CTU, 200 CTU, 250 CTU, 300 CTU, 400 CTU or 500 CTU cellulase per gram dry weight of solids in the slurry, or any range bounded by any two of the foregoing values, e.g., 10 to 200 CTU, 20 to 400 CTU, 40 to 250 CTU, etc.) and/or (ii) used singly or in enzyme/cocktail blends in doses ranging from 5 μg to 20 mg protein per gram dry weight of solids in the slurry (e.g., 5 μg, 10 μg, 50 μg, 100 μg, 250 μg, 500 μg, 1 mg, 2 mg, 5 mg, 10 mg, 20 mg, 30 mg, or 40 mg protein per gram dry weight of solids in the slurry, or any dosage range bounded by any two of the foregoing embodiments (e.g., 10 μg to 250 μg, from 20 μg to 500 μg, from 50 μg to 250 μg, from 10 μg to 100 μg, or from 20 μg to 250 μg, from 100 μg to 10 mg, from 250 μg to 20 mg, etc.);
(b) the reaction is carried out for a period of at least 0.25 minutes and optionally up to 48 hours (e.g., a time period of 0.25, 0.5, 1, 2, 5, 10, 15, 20, 25, 26, 27, 28, 29, or minutes or 0.5, 0.75, 1, 1.5, 2, 2.5, 3, 4, 5, 6, 12, 24, 36 or 48 hours, or for a time period ranging between any two of the foregoing values, e.g., 5 minutes to 1.5 hours, 10 minutes to 1 hour, 15 minutes to 2 hours, 2 minutes to 0.75 hour, 10 minutes to 0.75 hour, etc.);
(c) the reaction is carried out a pH between 3 and 6 (e.g., a pH of 3, 3.5, 4, 4.5, 5, 5.5 or 6, or at a pH ranging between any two of the foregoing values, e.g., 4-5.5 or 5-6, etc.);
(d) the reaction is carried out at a temperature of about 40° C. to about 80° C., 50° C. to about 80° C., about 40° C. to about 100° C., or even higher (e.g., up to about 90° C., 100° C., 110° C., or 120° C.) when using enzymes, such as PYROLASE (Verenium) that can withstand higher temperatures (e.g., a temperature of about 40° C., 45° C., 50° C., 55° C., 60° C., 62° C., 65° C., 70° C., 72° C., 75° C., 80° C., 90° C., 100° C., 110° C., or 120° C.), or at a temperature ranging between any two of the foregoing values, e.g., about 55° C. to 75° C., 60° C. to 80° C., 65° C. to 75° C., 60° C. to 70° C., 60° C. to 75° C., 65° C. to 80° C., 70° C. to 90° C., 50° C. to 100° C., 80° C. to 110° C., 65° C. to 75° C., 62° C. to 72° C., etc.);
(e) the slurry contains 5%-40% dry weight of biomass solids (e.g., 5%, 8%, 10%, 12%, 14%, 16%, 18%, 20%, 22%, 24%, 30%, 35% or 40% dry weight of biomass solids, and in some embodiments contains 15% or more and/or up to 25% dry weight of biomass solids, or contains solids in a range bounded by any two of the foregoing embodiments, such as, but not limited to, from 5% to 25%, from 8% to 20%, from 10% to 22%, from 12% to 24%, from 14% to 24%, from 15% to 25%, from 16% to 24%, from 16% to 22%, from 18% to 22%, from 16% to 30%, from 14% to 30% dry weight biomass solids, etc.);
(f) the biomass has been subject to acid (e.g., sulfuric, nitric, acetic or phosphoric acid) pretreatment; and/or
(g) the biomass has been subject to steam explosion, for example under:
   (i) a pressure of 50-400 psig, 50-300 psig, 50-250 psig, 75-200 psig, 75-150 psig, 100-200 psig, 100-250 psig, or 150-250 psig, and/or
   (ii) a temperature of 105-300° C., 105-210° C., 150-250° C., 190-210° C., or 190-250° C. and/or
   (iii) for a time period ranging from 0.1-10 minutes, 0.25-8 minutes, from 0.5-2 minutes or from 1-5 minutes, or
   (iv) under conditions bounded by any two pressure, two temperature and/or two time embodiments identified above, for example (i) a pressure of 100-300 psig or 75-250 psig and/or (ii) a temperature of 105-210° C. or 150-210° C. and/or (iii) a time period of 0.1-2 minutes or 0.25-5 minutes or 1-8 minutes.

In some embodiments, a liquefaction reaction of the disclosure is characterized by two, three, four, five, six or all seven of features (a) through (g) above. In some exemplary embodiments, a liquefaction reaction of the disclosure is characterized by a combination of selected from the following table:

| Exemplary | Required | |
|---|---|---|
| Embodiment | Features | Optional Features |
| A | (a), (b) | One, two, three, four or all five of (c), (d), (e), (f) and (g) |
| B | (a), (c) | One, two, three, four or all five of (b), (d), (e), (f), and (g) |
| C | (a), (d) | One, two, three, four or all five of (b), (c), (e), (f), and (g) |
| D | (a), (e) | One, two, three, four or all five of (b), (c), (d), (f), and (g) |
| E | (a), (f) | One, two, three, four or all five of (b), (c), (d), (e), and (g) |
| F | (a), (g) | One, two, three, four or all five of (b), (c), (d), (e), and (f) |
| G | (b), (c) | One, two, three, four or all five of (a), (d), (e), (f), and (g) |
| H | (b), (d) | One, two, three, four or all five of (a), (c), (e), (f), and (g) |
| I | (b), (e) | One, two, three, four or all five of (a), (c), (d), (f), and (g) |
| J | (b), (f) | One, two, three, four or all five of (a), (c), (d), (e), and (g) |
| K | (b), (g) | One, two, three, four or all five of (a), (c), (d), (e), and (f) |
| L | (c), (d) | One, two, three, four or all five of (a), (b), (e), (f), and (g) |
| M | (c), (e) | One, two, three, four or all five of (a), (b), (d), (f), and (g) |
| N | (c), (f) | One, two, three, four or all five of (a), (b), (d), (e), and (g) |
| O | (c), (g) | One, two, three, four or all five of (a), (b), (d), (e), and (f) |
| P | (d), (e) | One, two, three, four or all five of (a), (b), (c), (f), and (g) |
| Q | (d), (f) | One, two, three, four or all five of (a), (b), (c), (e), and (g) |
| R | (d), (g) | One, two, three, four or all five of (a), (b), (c), (e), and (f) |
| S | (e), (f) | One, two, three, four or all five of (a), (b), (c), (d), and (g) |
| T | (e), (g) | One, two, three, four or all five of (a), (b), (c), (d), and (f) |
| U | (f), (g) | One, two, three, four or all five of (a), (b), (c), (d), and (e) |

Additional embodiments that can be used in connection with the methods and compositions of the present disclosure, optionally in conjunction with one or more of embodiments A-U above, can be found in Section 7 below.

The biomass is preferably lignocellulosic and can include, without limitation, seeds, grains, tubers, industrial/consumer waste materials that are rich in cellulose, hemicellulose and/or pectin, plant waste or byproducts of food processing or industrial processing (e.g., stalks), corn (including, e.g., cobs, stover, and the like), energy crops and agricultural residues, forestry residues, grasses (including, but not limited to, e.g., Napier Grass or Uganda Grass, such as Pennisetum purpureum; or, Miscanthus; such as Miscanthus giganteus and other varieties of the genus Miscanthus, or Indian grass, such as Sorghastrum nutans; or, switchgrass, e.g., as Panicum virgatum or other varieties of the genus Panicum, giant reed, e.g., as arundo donax or other varieties of the genus arundo, energy cane e.g., as saccharum pp.), wood (including, e.g., wood chips, processing waste)), paper, pulp, and recycled paper (including, e.g., newspaper, printer paper, and the like). In some embodiments the biomass is energy cane or sugarcane, which refers to any species of tall perennial grasses of the genus Saccharum. Other biomass materials include, without limitation, potatoes, soybean (e.g., rapeseed), barley, rye, oats, wheat, beets, sorghum sudan, milo, bulgher, rice, and sugar cane bagasse. Further sources of biomass are disclosed in Section 5.1 and can be used in the present methods.

Suitable ratios of biomass and the aqueous liquid in the biomass slurries of the disclosure are at a 1:1 to 1:7, 1:2 to 1:6, 1:1 to 1:5.7, 1:2 to 1:6, 1:2.5 to 1:5.7, 1:3.33 to 1:5.7, or 1:4 to 1:5.7 solid:liquid weight ratio, or in a solid:liquid weight ratio bounded by any two of the foregoing embodiments, for example 1:2-1.3.33, 1:1-1:2, or 1:2.5-1:7.

Hydrolyzing proteins refers to cellulase enzymes, hemicellulase enzymes and/or accessory proteins and enzymes that can participate (directly or indirectly) in the digestion of lignocellulosic biomass into sugar monomers or oligomers. Cellulases include exo-acting cellobiohydrolases (CBHs), endoglucanases (EGs) and β-glucosidases (BGs). Many plants and microorganisms produce cellulase cocktails, which can include accessory proteins. For example, the cellulase cocktail produced by Trichoderma reesei can include the CBH I (more generally, Cel7A), CBH2 (Cel6A), EG1 (Cel7B), EG2 (Cel5), EG3 (Cel12), EG4 (Cel61A), EG5 (Cel45A), EG6 (Cel74A), Cip1, Cip2, β-glucosidases (including, e.g., Cel3A), acetyl xylan esterase, β-mannanase, and swollenin. Further information regarding hydrolyzing proteins can be found in Section 5.4.

The term CTU as used herein refers to units of cellulase activity as measured using CELLAZYME T tablets (Megazyme, Co. Wickow, Ireland). The substrate in this assay is azurine-crosslinked Tamarind Xyloglucan (AZCL-Xyloglucan). This substrate is prepared by dyeing and cross-linking highly purified xyloglucan to produce a material which hydrates in water but is water insoluble. Hydrolysis by cellulases produces water soluble dyed fragments and the rate of release of these (increase in absorbance at 590 nm) can be related directly to enzyme activity. One CTU is defined as the amount of enzyme required to release one micromole of glucose reducing sugar-equivalents per minute from barley β-glucan (10 mg/ml) at pH 4.5 and 40° C. 7.5 CTUs of cellulase cocktail corresponds to approximately 1 filter paper unit (“FPU”). As used herein, the term FPU refers to filter paper units as determined by the method of Adney and Baker, Laboratory Analytical Procedure #006 (“LAP-006”), “Measurement of cellulase activity,” Aug. 12, 1996, the USA National Renewable Energy Laboratory (NREL), which is expressly incorporated by reference herein in entirety. 1 mg of total protein of a T. reesei cellulase cocktail (as measured by the Bradford assay) corresponds to approximately 27.4 CTU. In alternative embodiments of the liquefaction methods, the reference to enzyme dosages in “CTUs” can be replaced with the approximate corresponding amount of enzyme by protein mass or FPUs, using the conversion of 36.5 μg of a cellulase or cellulase cocktail or 0.133 FPU of a cellulase or cellulase cocktail per CTU. Accordingly, in these alternative embodiments, enzyme dosages referred to by CTUs in the various aspects of the disclosure are substituted by the corresponding dosage in protein mass or FPU. Thus, for example, alternatives to an embodiment of the liquefaction methods in which the enzyme dose is 20 to 400 CTU are embodiments in which the enzyme dose is 730 μg to 14.6 mg protein or a cellulase or cellulase cocktail characterized by an activity of 2.67 to 53.33 FPU.

The biomass can be pretreated, for example by steam explosion and/or with an acid (e.g., sulfuric acid) or a base (e.g., ammonia), prior to liquefaction. If the biomass is subject to both steam explosion and acid pretreatment, the steam explosion can precede or follow the acid pretreatment. Suitable pretreatment methods are described in Section 5.2.

A liquefaction reaction can be carried out as a batch process, as a continuous process, or as a semi-continuous process. The process can be carried out at large scales, for example in volumes of at least 10 liters, at least 20 liters, at least 50 liters, at least 100 liters, at least 250 liters, at least 500 liters, at least 1,000 liters, or at least 5,000 liters, for example 2,000 liters, 5,000 liters 10,000 liters, 25,000 liters, 50,000 liters, 100,000 liters, 250,000 liters, 500,000 liters, or 1,000,000 liters or more. In specific embodiments, the liquefaction reaction volume is in a range bounded by any two of the foregoing values, e.g., 10 liters to 2,000 liters, 50 liters to 20,000 liters, 250 liters to 25,000 liters, 250 liters to 1,000 liters, 250 liters to 5,000 liters, 500 liters to 10,000 liters, 1,000 liters to 50,000 liters, 5,000 liters to 25,000 liters, 1,000 liters to 100,000 liters, 100,000 liters to 500,000 liters, 50,000 to 1,000,000 liters, or 250,000 liters to 1,000,000 liters, etc. The process can also be carried out in volumes of at least 10 gallons, at least 20 gallons, at least 50 gallons, at least 100 gallons, at least 250 gallons, at least 500 gallons, at least 1,000 gallons, or at least 5,000 gallons, for example 2,000 gallons, 5,000 gallons 10,000 gallons, 25,000 gallons, 50,000 gallons, 100,000 gallons, 250,000 gallons, 500,000 gallons, or 1,000,000 gallons or more. In specific embodiments, the liquefaction reaction volume is in a range bounded any two of the foregoing values, e.g., 10 gallons to 2,000 gallons, 50 gallons to 20,00 gallons, 250 gallons to 25,000 gallons, 250 gallons to 1,000 gallons, 250 gallons to 5,000 gallons, 500 gallons to 10,000 gallons, 1,000 gallons to 50,000 gallons, 5,000 gallons to 25,000 gallons, 1,000 gallons to 100,000 gallons, 100,000 gallons to 500,000 gallons, 50,000 to 1,000,000 gallons, or 250,000 liters to 1,000,000 gallons, etc.

The liquefaction reactions advantageously permit continuous reactions to proceed without intermittently stopping and cleaning reaction vessels. In some embodiments, the reactions proceed for periods of at least one day, at least 2 days, at least 3 days, at least 4 days, at least 5 days, at least 6 days, at least a week, weeks, at least a month, months, at least a year, years or more. In some embodiments, reaction proceeds continuously without stopping. In continuous mode, the retention time or residence time in the vessel is preferably 2 hours or less (e.g., a time period of 0.5, 1, 2, 5, 10, 15, 20, 25, 26, 27, 28, 29, or 30 minutes or 0.5, 0.75, 1, 1.5 or 2 hours, or for a time period ranging between any two of the foregoing values, e.g., 5 minutes to 1.5 hours, 10 minutes to 1 hour, 15 minutes to 2 hours, 2 minutes to 0.75 hour, 10 minutes to 0.75 hour, etc.).

For liquefaction reactions carried out at temperatures greater than 55° C. or greater than 60° C., it can be advantageous to heat the one or more hydrolyzing proteins (e.g., cellulases) in the presence of biomass solids for maximum viscosity reduction.

In various embodiments, the methods of the disclosure result in a viscosity reduction of a biomass by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or at least 85% as compared to the slurry viscosity prior to hydrolyzing protein treatment. Liquefaction results in the use of less power to agitate a liquefied slurry. Accordingly, in some embodiments, reduction in power usage, as indicated by a variable such as current, can be used as a surrogate for viscosity reduction. When using a reduction in power usage as a surrogate for viscosity reduction, the methods of the disclosure reduce the amount of power (e.g., as indicated by current) required to agitate the slurry by at least 10%, at least 20%, at least 30%, at least 40%, or at least 50% as compared to power usage in the absence of enzymatic treatment. The current measurement can be carried out 2 minutes to 2 hours after the treatment, e.g., 10 minutes, 20 minutes or 30 minutes after the treatment.

The liquefaction methods disclosed herein are preferably carried out under conditions that result in minimal saccharification, e.g., 10% or less of the theoretical yield of glucose, xylose and/or cellobiose. In certain embodiments, the extent of saccharification is 8% or less, 6% or less, 5% or less, 4% or less, 3% or less, or 2% or less of the theoretical yield of (i) glucose, (ii) xylose, (iii) cellobiose, (iv) both glucose and xylose, (v) both glucose and cellobiose, (vi) both xylose and cellobiose, or (vii) each of glucose, xylose and cellobiose.

The methods of the disclosure can include further steps in addition to liquefaction, such one or more steps depicted in FIG. 1A or FIG. 1B that are upstream or downstream of the liquefaction step (3). For example, the methods can include a pretreatment step (1), optionally with liquids/solids separation (2), prior to liquefaction (see Section 5.2). The solids can be further processed, for example in a screw press, prior to slurry formation and liquefaction (see Section 5.2). The methods can include a saccharification step and optionally a fermentation step (see Sections 5.5 and 5.6) without or without a product recovery step (see Section 5.7) downstream of the liquefaction step. The saccharification and fermentation can be carried out separately ((4a) and (4b) in FIG. 1A) or simultaneously ((4) in FIG. 1B), optionally in a consolidated bioprocessing method. The resulting fermentation product can be recovered/isolated (5). The recovered fermentation product can be further processed (6), e.g., dehydrated, and the waste product (e.g., stillage), processed, for example by a solids/liquids separation step, e.g., centrifugation (7).

The present inventors have discovered that the solids/liquids separation step requires the addition of less (or even no) water when using biomass liquefied with a hydrolyzing enzyme than when using non-liquefied biomass. Accordingly, the present disclosure further provides methods in which the liquefaction step is followed by simultaneous or separate saccharification and fermentation, recovery of the fermentation product (e.g., ethanol), and processing the waste product (e.g., solids/liquids separate of stillage, for example by centrifugation) in a process that includes that addition of at least 10%, at least 20%, at least 30%, at least 40%, at least 50%, at least 60%, at least 70% or at least 80% less water, e.g., prior to centrifugation, than would be added in a comparable process in which the biomass is not subject to a liquefaction step. In some embodiments, no water is added during solids/liquids separation of stillage.

### 4. BRIEF DESCRIPTION OF THE FIGURES AND TABLES

FIGS. 1A-1B: Schematic depiction of biofuels production processes including a liquefaction step. FIG. 1A: Generation of fermentation products using separate saccharification and fermentation processes. FIG. 1B: Generation of fermentation products using simultaneous saccharification and fermentation processes.

FIG. 2: Schematic of a continuous stirred tanked reactor.

FIGS. 3A-3B: Liquefaction of alkaline pretreated pine and eucalyptus pulps by cellulase. FIG. 3A: untreated samples. FIG. 3B: samples treated with three doses of enzyme. E=eucalyptus; RP=radiata pine; MP or M=mixed pine.

FIG. 4: Increase in motor current in SSF reaction tank with increasing weight percentage of solids in biomass slurry.

FIG. 5: Motor current over the course of a SSF reaction.

FIGS. 6A-6B: FIG. 6A: Schematic of viscometer (Perten Rapid Visco Analyzer) used to analyze biomass liquefaction. FIG. 6B: Liquefaction of 14% sugar cane pretreated limed slurry at different temperatures.

FIG. 7: Liquefaction of 14% sugar cane H₂SO₄-pretreated limed slurry at different temperatures close up. Spikes in viscosity are the result of fibers and clumps catching on the spindle.

FIG. 8: Enzyme dosing at 70° C.

FIG. 9: Close up of 70° C. dosing application of FIG. 8.

FIGS. 10A-10B: FIG. 10A: enzyme addition and slurry dilution water stepped down to 0.75× while maintaining pump current below 20 amps. FIG. 10B: no enzyme addition and slurry dilution water flow rate at 1× to maintain pump current draw below 20 amps.

FIGS. 11A-11C: Viscosity as a function of time. FIG. 11A: viscosity time course at 100 rpm. FIG. 11B: viscosity time course at 20 rpm. FIG. 11C: viscosity time course at 3 rpm. The initial viscosity measurement (▴) and averaged steady-state measurements (⋄) are shown.

FIGS. 12A-12B: Enzymatic viscosity reduction of pretreated sorghum at 18% solids, 50° C., pH5.4 over 30 minutes at rotational speeds between 2-100 rpm. FIG. 12A: initial viscosity measurements at each time point. FIG. 12B: averaged steady-state viscosity measurements at each time point.

FIG. 13: Percent reduction in viscosity measured in an 18% solids slurry of sorghum at 50° C., pH5.4, with 25 CTU Kerry Biocellulase W/gram solids enzyme load.

FIG. 14: Percent decrease between initial viscosity measurement and steady-state, averaged over six time points (0, 60, 300, 600, 900, and 1800 seconds), for each rotational speed.

FIGS. 15A-15E: Photographs of pretreated cakes. FIG. 15A: A1 and A2 cakes. FIG. 15B: B1 and B2 cakes. FIG. 15C: C1 and C2 cakes. FIG. 15D: D1 and D2 cakes. FIG. 15E: E1 and E2 cakes. Sample nomenclature is as defined in Table 7.

FIGS. 16A-16B: Viscosity as a function of time for A1 (FIG. 16A) and A2 (FIG. 16B), 10% solids at 20 rpm. Sample nomenclature is as defined in Table 7.

FIGS. 17A-17D: Viscosity reduction of sulfuric acid pretreated samples. FIG. 24A: 10% solids at 20 rpm for unexploded vs. steam exploded cake. FIG. 24B: 5% solids at 3 rpm for unexploded vs. steam exploded cake. FIG. 24C: 10% solids at 20 rpm for steam-exploded cake, no enzyme vs. 25 CTU/g solids enzyme load. FIG. 24D: 10% solids at 3 rpm for steam-exploded cake, no enzyme, 25 CTU/g, and 50 CTU/g enzyme load.

FIGS. 18A-18B: Viscosity as a function of time for B1 (FIG. 18A) and B2 (FIG. 18B), 10% solids at 20 rpm. Sample nomenclature is as defined in Table 7.

FIGS. 19A-19D: Viscosity reduction of nitric acid pretreated samples. FIG. 19A: 10% solids at 20 rpm for unexploded vs. steam exploded cake. FIG. 19B: 13% solids at 3 rpm for unexploded vs. steam exploded cake. FIG. 19C: 10% solids at 20 rpm for steam-exploded cake, no enzyme vs. 25 CTU/g solids enzyme load. FIG. 19D: 10% solids at 3 rpm for steam-exploded cake, no enzyme vs. 25 CTU/g solids enzyme load.

FIGS. 20A-20B: Viscosity as a function of time for C1 (FIG. 20A) and C2 (FIG. 20B), 5% solids at 20 rpm. Sample nomenclature is as defined in Table 7.

FIGS. 21A-21B: Viscosity as a function of time for D1 (FIG. 21A) and D2 (FIG. 21B), 5% solids at 20 rpm. Sample nomenclature is as defined in Table 7.

FIGS. 22A-22B: Viscosity as a function of time for E1 (FIG. 22A) and E2 (FIG. 22B), 5% solids at 20 rpm. Sample nomenclature is as defined in Table 7.

FIGS. 23A-23C: Viscosity reduction of pretreated samples (5% solids at 20 rpm) for unexploded vs. steam exploded cake with 25 CTU/g solids enzyme loading. FIG. 23A: phosphoric acid pretreatment. FIG. 23B: acetic acid pretreatment. FIG. 23C: autohydrolysis.

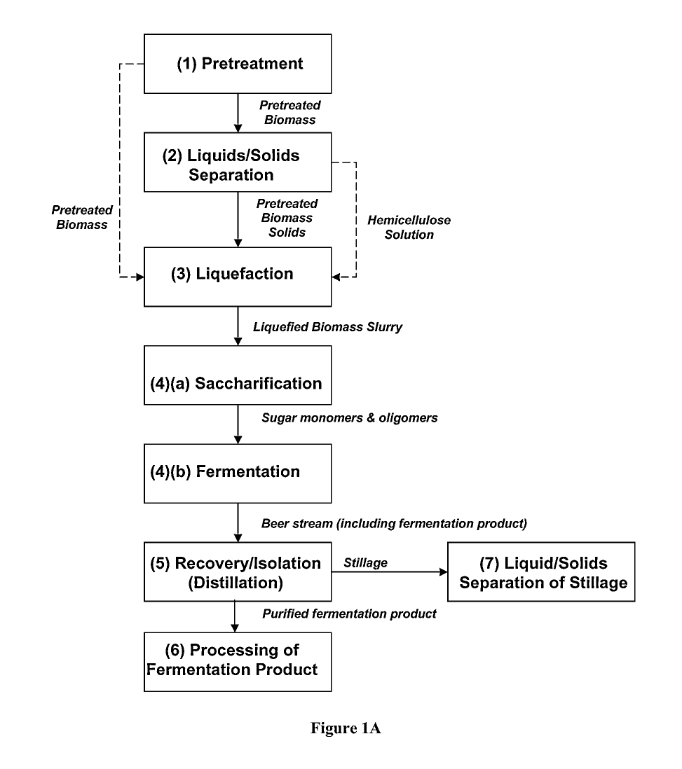

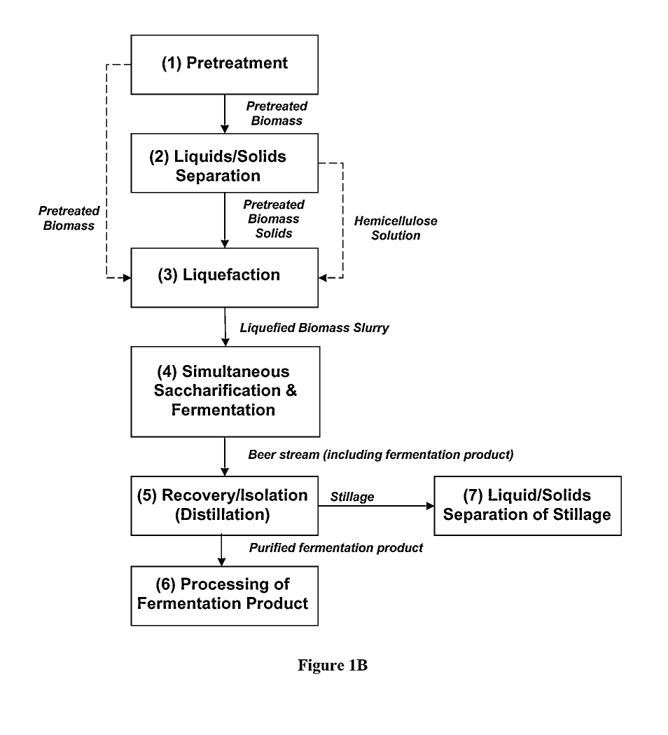

Table 1: Degree of saccharification of alkaline pretreated pine and eucalyptus pulps of treated with cellulase.

Table 2: Sugar content and viscosity of liquefied biomass.

Table 3: Reduction in viscosity of biomass slurry treated with cellulase at 60° C.

Table 4: Reduction in viscosity of biomass slurry treated with cellulase at 70° C.

Table 5: (A) Initial and (B) Average steady-state viscosity of 18% solids pretreated sorghum at selected timepoints for each rotational speed.

Table 6: Hydrolysis and steam-explosion conditions used for pretreatment of Napier grass samples.

Table 7: Compositional analysis of unexploded (1-series) and steam-exploded (2-series) cakes pretreated with various acids. All references to samples shall be in accordance with the nomenclature of Table 7 unless indicated otherwise.

Table 8: Bauer-McNett fiber classification of pretreated Napier grass samples.

Table 9: MorFi fiber analysis of pretreated Napier grass samples.

Table 10: Data from viscosity analysis. Time constants are defined as the time required to bring the viscosity to within 63% of the equilibrium viscosity value. Three time constants would be within 99% of the equilibrium value.

### 5. DETAILED DESCRIPTION

The present disclosure relates to compositions and methods for biomass liquefaction. The methods of the disclosure generally entail subjecting biomass slurry to one or more hydrolyzing proteins and/or forming a biomass slurry in the presence of one or more hydrolyzing proteins. Types of biomass that can be used in the present methods include but are not limited to those described in Section 5.1. The biomass is preferably pretreated. Exemplary methods of pretreatment are described in Section 5.2. Methods of biomass liquefaction are described in Section 5.3, and hydrolyzing proteins suitable for use in the liquefaction methods are described in Section 5.4. Following liquefaction, the biomass can be saccharified (for example as described in Section 5.5) and optionally used to manufacture biobased products by fermentation or chemical synthesis (for example as described in Section 5.6). The resulting fermentation products can be recovered (for example as described in Section 5.7). The use of liquefied biomass in fermentation reactions permits more efficient waste treatment processes, e.g., as described in Section 5.8.

5.1. Biomass

The term “biomass,” as used herein, refers to any composition comprising cellulose (optionally also hemicellulose and/or lignin).

Relevant types of biomasses for liquefaction according to the present invention can include biomasses derived from agricultural crops such as, e.g., containing grains; corn stover, grass, bagasse, straw e.g. from rice, wheat, rye, oat, barley, rape, sorghum; tubers. e.g., beet and potato.

Relevant types of lignocellulosic biomasses for liquefaction according to the present invention can include biomasses from the grass family. The proper name is the family known as Poaceae or Gramineae in the class Liliopsida (the monocots) of the flowering plants. Plants of this family are usually called grasses, and include bamboo. There are about 600 genera and some 9,000-10,000 or more species of grasses (Kew Index of World Grass Species).

Poaceae includes the staple food grains and cereal crops grown around the world, lawn and forage grasses, and bamboo.

The success of the grasses lies in part in their morphology and growth processes, and in part in their physiological diversity. Most of the grasses divide into two physiological groups, using the C3 and C4 photosynthetic pathways for carbon fixation. The C4 grasses have a photosynthetic pathway linked to specialized leaf anatomy that particularly adapts them to hot climates and an atmosphere low in carbon dioxide. C3 grasses are referred to as “cool season grasses” while C4 plants are considered “warm season grasses”.

Grasses may be either annual or perennial. Examples of annual cool season are wheat, rye, annual bluegrass (annual meadowgrass, Poa annua and oat). Examples of perennial cool season are orchardgrass (cocksfoot, Dactylis glomerata), fescue (Festuca spp.), Kentucky Bluegrass and perennial ryegrass (Lolium perenne). Examples of annual warm season are corn, sudangrass and pearl millet. Examples of Perennial Warm Season are big bluestem, indiangrass, bermudagrass and switchgrass.

One classification of the grass family recognizes twelve subfamilies: These are 1) anomochlooideae, a small lineage of broad-leaved grasses that includes two genera (Anomochloa, Streptochaeta); 2) Pharoideae, a small lineage of grasses that includes three genera, including Pharus and Leptaspis; 3) Puelioideae a small lineage that includes the African genus Puelia; 4) Pooideae which includes wheat, barely, oats, brome-grass (Bronnus) and reed-grasses (Calamagrostis); 5) Bambusoideae which includes bamboo; 6) Ehrhartoideae, which includes rice, and wild rice; 7) Arundinoideae, which inludes the giant reed and common reed 8) Centothecoideae, a small subfamily of 11 genera that is sometimes included in Panicoideae; 9) Chloridoideae including the lovegrasses (Eragrostis, ca. 350 species, including teff), dropseeds (Sporobolus, some 160 species), finger millet (Eleusine coracana (L.) Gaertn.), and the muhly grasses (Muhlenbergia, ca. 175 species); 10) Panicoideae including panic grass, maize, sorghum, sugar cane, most millets, fonio and bluestem grasses; 11) Micrairoideae; 12) Danthoniodieae including pampas grass; with Poa which is a genus of about 500 species of grasses, native to the temperate regions of both hemisphere.

Agricultural grasses grown for their edible seeds are called cereals. Three common cereals are rice, wheat and maize (corn). Of all crops, 70% are grasses.

Therefore a preferred biomass is selected from the group consisting of the eneregy crops. In a further preferred embodiment, the energy crops are grasses. Preferred grasses include Napier Grass or Uganda Grass, such as Pennisetum purpureum; or, Miscanthus; such as Miscanthus giganteus and other varieties of the genus Miscanthus, or Indian grass, such as Sorghastrum nutans; or, switchgrass, e.g., as Panicum virgatum or other varieties of the genus Panicum, giant reed, e.g., as arundo donax or other varieties of the genus arundo, energy cane e.g., as saccharum pp.). In some embodiments the biomass is sugarcane, which refers to any species of tall perennial grasses of the genus Saccharum.

Other types of biomass suitable for liquefaction according to the present methods include seeds, grains, tuber (e.g., potatoes and beets), plant waste or byproducts of food processing or industrial processing (e.g., stalks), corn and corn byproducts (including, e.g., corn husks, corn cobs, corn fiber, corn stover, and the like), wood and wood byproducts (including, e.g., processing waste, deciduous wood, coniferous wood, wood chips (e.g., deciduous or coniferous wood chips), sawdust (e.g., deciduous or coniferous sawdust)), paper and paper byproducts (e.g., pulp, mill waste, and recycled paper, including, e.g., newspaper, printer paper, and the like), soybean (e.g., rapeseed), barley, rye, oats, wheat, beets, sorghum sudan, milo, bulgher, rice, sugar cane bagasse, forest residue, agricultural residues, quinoa, wheat straw, milo stubble, citrus waste, urban green waste or residue, food manufacturing industry waste or residue, cereal manufacturing waste or residue, hay, straw, rice straw, grain cleanings, spent brewer's grain, rice hulls, salix, spruce, poplar, eucalyptus, Brassica carinata residue, Antigonum leptopus, sweetgum, Sericea lespedeza, Chinese tallow, hemp, rapeseed, Sorghum bicolor, soybeans and soybean products (soybean leaves, soybeans stems, soybean pods, and soybean residue), sunflowers and sunflower products (e.g., leaves, sunflower stems, seedless sunflower heads, sunflower hulls, and sunflower residue), Arundo, nut shells, deciduous leaves, cotton fiber, manure, coastal Bermuda grass, clover, Johnsongrass, flax, straw (e.g., barley straw, buckwheat straw, oat straw, millet straw, rye straw amaranth straw, spelt straw), amaranth and amaranth products (e.g., amaranth stems, amaranth leaves, and amaranth residue), alfalfa, and bamboo.

Yet further sources of biomass include hard wood and soft wood.

Examples of suitable softwood trees include, but are not limited to, the following: pine trees, such as loblolly pine, jack pine, Caribbean pine, lodgepole pine, shortleaf pine, slash pine, Honduran pine, Masson's pine, Sumatran pine, western white pine, egg-cone pine, logleaf pine, patula pine, maritime pine, ponderosa pine, Monterey pine, red pine, eastern white pine, Scots pine, araucaria tress; fir trees, such as Douglas fir; and hemlock trees, plus hybrids of any of the foregoing.

Examples of suitable hardwood trees include, but are not limited to, the following: eucalyptus trees, such as Dunn's white gum, Tasmanian bluegum, rose gum, Sydney bluegum, Timor white gum, and the E. urograndis hybrid; populus trees, such as eastern cottonwood, bigtooth aspen, quaking aspen, and black cottonwood; and other hardwood trees, such as red alder, Sweetgum, tulip tree, Oregon ash, green ash, and willow, plus hybrids of any of the foregoing.

5.2. Pretreatment

Lignocellulosic fibers comprise a complex network of cellulose, hemicellulose and lignin in a compact matrix that is difficult to hydrolyze due to poor enzyme accessibility. To improve accessibility of enzymes to the interwoven polysaccharides, a mechanical, thermal and/or chemical (e.g., a thermomechanochemical) “pretreatment” is typically necessary before enzymatic hydrolysis in order to render cellulose material more accessible or susceptible to enzymes and thus more amenable to hydrolysis into simple sugars.

Any pretreatment process can be used to prepare lignocellulosic biomass for liquefaction. Acid hydrolysis is a cheap and fast method and can suitably be used. A concentrated acid hydrolysis is preferably operated at temperatures from 20° C. to 100° C., and an acid strength in the range of 10% to 45% (e.g., 10%, 10.5%, 11%, 11.5%, 12%, 12.5%, 13%, 13.5%, 14%, 14.5%, 15%, 15.5%, 16%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 37%, 37.5%, 38%, 38.5%, 39%, 39.5%, 40%, 41%, 41.5%, 42%, 42.5%, 43%, 43.5%, 44%, 44.5%, 45% or any range bounded by any two of the foregoing values). Dilute acid hydrolysis is a simpler process, but is optimal at higher temperatures (100° C. to 230° C.) and pressure. Different kinds of acids, with concentrations in the range of 0.001% to 10% (e.g., 0.001%, 0.01%, 0.05%, 0.1%, 0.15%, 0.2%, 0.25%, 0.3%, 0.35%, 0.4%, 0.5%, 0.6%, 0.7%, 0.8%, 0.9%, 1%, 2%, 3%, 4%, 5%, 5.5%, 6%, 6.5%, 7%, 7.5%, 8%, 8.5%, 9%, 9.5% or 10%, or any range bounded by any two of the foregoing values) are preferably used. Suitable acids include nitric acid, sulfurous acid, nitrous acid, phosphoric acid, acetic acid, hydrochloric acid and sulfuric acid can be used in the pretreatment step. Preferably sulfuric acid is used.

Depending on the acid concentration, and the temperature and pressure under which the acid pretreatment step is carried out, corrosion resistant equipment and/or pressure tolerant equipment may be needed.

The pretreatment can be carried out for a time period ranging from 2 minutes to 10 hours (e.g., 2, 3, 4, 5, 6, 7, 8, 9, 10, 15, 20, 25, 26, 27, 28, 29, or 30 minutes, or 0.5, 0.75, 1, 1.5, 2, 2.5, 3, 3.5, 4, 4.5, 5, 5.5, 6, 6.5, 7, 7.5, 8, 8.5, 9, 9.5 or 10 hour, or range bounded by any two of the foregoing values), preferably 1 minute to 2 hours, 2 minutes to 15 minutes, 2 minutes to 2 hours, 15 minutes to 2 hours, 30 minutes to 2 hours, or 10 minutes to 1.5 hours.

Variations of acid pretreatment methods are known in the art and are encompassed by the methods of the present disclosure. A preferred pretreatment method entails hydrolyzing biomass by subjecting the biomass material to a first (chemical) hydrolysis step in an aqueous medium at a temperature and a pressure chosen to effectuate primarily depolymerization of hemicellulose without achieving significant depolymerization of cellulose into glucose. This step yields a slurry in which the liquid aqueous phase contains dissolved monosaccharides and soluble and insoluble oligomers of hemicellulose resulting from depolymerization of hemicellulose, and a solid phase containing cellulose and lignin. See, e.g., U.S. Pat. No. 5,536,325. In a preferred embodiment, sulfuric acid is utilized to effect the first hydrolysis step.

In another embodiment, the pretreatment entails subjecting biomass material to a catalyst comprising a dilute solution of a strong acid and a metal salt in a reactor. The biomass material can, e.g., be a raw material or a dried material. This pretreatment can lower the activation energy, or the temperature, of cellulose hydrolysis, ultimately allowing higher yields of fermentable sugars. See, e.g., U.S. Pat. Nos. 6,660,506; 6,423,145.

A further exemplary method involves processing a biomass material by one or more stages of dilute acid hydrolysis using about 0.4% to about 2% of an acid; followed by treating the unreacted solid lignocellulosic component of the acid hydrolyzed material with alkaline delignification. See, e.g., U.S. Pat. No. 6,409,841. Another exemplary pretreatment method comprises prehydrolyzing biomass (e.g., lignocellulosic materials) in a prehydrolysis reactor; adding an acidic liquid to the solid lignocellulosic material to make a mixture; heating the mixture to reaction temperature; maintaining reaction temperature for a period of time sufficient to fractionate the lignocellulosic material into a solubilized portion containing at least about 20% of the lignin from the lignocellulosic material, and a solid fraction containing cellulose; separating the solubilized portion from the solid fraction, and removing the solubilized portion while at or near reaction temperature; and recovering the solubilized portion. The cellulose in the solid fraction is rendered more amenable to enzymatic digestion. See, e.g., U.S. Pat. No. 5,705,369. Further pretreatment methods can involve the use of hydrogen peroxide H₂O₂. See Gould, 1984, Biotech, and Bioengr. 26:46-52.

The pretreatment can also include, as an alternative (e.g., in the absence of) or in addition to (e.g., before or after) the acid treatment, a heat or pressure treatment or a combination of heat and pressure, e.g., treatment with steam, for about 0.5 hours to about 10 hours (e.g., 0.5, 1, 1.5, 2, 2.5, 3, 3.5, 4, 4.5, 5.5, 6, 6.5, 7, 7.5, 8, 8.5, 9, 9.5 or 10 hours, or any range bounded by any two of the foregoing values). The steam treatment can also include a steam explosion, which couples the steam pretreatment with an explosive discharge of the material after the pretreatment. Steam explosion generally involves a rapid flashing of material to a lower pressure, either atmospheric, negative or positive pressure, producing turbulent flow of the material to increase the accessible surface area by fragmentation. Any steam explosion method known in the art can be used herein, for example as described in Duff and Murray, 1996, Bioresource Technology 855: 1-33; Galbe and Zacchi, 2002, Appl. Microbiol. Biotechnol. 59: 618-628; U.S. Patent Publication No. 2002/0164730; U.S. Patent Publication No. 2012/0104313; U.S. Patent Publication No. 2012/0111515; U.S. Patent Publication No. 2008/0277082; and U.S. Patent Publication No. 2009/0221814.

The steam explosion step can be carried out in a steam digester, which is also known in the art. For example, feedstock having a moisture content of about 45% to about 55% by weight may be fed to an autohydrolysis digester wherein the biomass is hydrolyzed under steam at high pressure (e.g., 50-400 psig, more preferably 50-300 psig, 50-250 psig, 75-200 psig or 75-150 psig) and temperature (e.g., 105-300° C., 150-250° C. or 190° C.-210° C.) for a time period typically ranging from about 10 seconds to about 10 minutes (for example from 30 seconds to about 2-5 minutes, optionally in the presence of a catalyst, such as sulfuric acid. At the instant of release from the digester (steam explosion), the biomass exits the high temperature, high pressure hydrolyzer into a reduced pressure, for which can be greater or equal to atmospheric pressure or even a vacuum. The pressure in the digester is typically released suddenly, e.g., in less than 2 second, less than 1 second or even instantaneously. The rapid decrease in pressure results in the biomass separating into individual fibers or bundles of fibers.

Biomass can also be treated by atmospheric-pressure (AP) plasma as an alternative to steam explosion. See, e.g., U.S. Patent Publication No. 2008/0006536.

Pretreatment can also comprise contacting a biomass material with stoichiometric amounts of sodium hydroxide and ammonium hydroxide at a very low concentration. See Teixeira et al., 1999, Appl. Biochem. and Biotech. 77-79:19-34. Pretreatment can also comprise contacting a lignocellulose with a chemical (e.g., a base, such as sodium carbonate or potassium hydroxide) at a pH of about 9 to about 14 at moderate temperature, pressure, and pH. See PCT Publication WO2004/081185.

Ammonia pretreatment can also be used. Such a pretreatment method comprises subjecting a biomass material to low ammonia concentration under conditions of high solids. See, e.g., U.S. Patent Publication No. 20070031918 and PCT publication WO 06/110901.

Following pretreatment, the pretreated product comprises a mixture of acid or base, partially degraded biomass and fermentable sugars. Prior to further processing, the acid or base can be removed from the pretreated biomass by applying a vacuum. The pretreated biomass can also be neutralized prior to liquefaction.

The entire pretreatment mixture comprising both soluble and insoluble fractions can subject to liquefaction as described in Section 5.3. Alternatively, the aqueous fraction comprising the solubilized sugars (typically hemicellulases) can be separated from insoluble particulates remaining in the mixture. Methods for separating the soluble from the insoluble fractions (i.e., the pretreated biomass solids) include, but are not limited to, decantation and filtration. The pretreated biomass solids can optionally be washed with an aqueous solvent (e.g., water) to remove adsorbed sugars prior to liquefaction. The soluble fraction can also be included in a liquefaction reaction, and is optionally concentrated prior to liquefaction using a suitable process, such as evaporation.

The solids can be further processed prior to liquefaction, for example dewatered. Dewatering can be suitably achieved with a screw press. The screw press is a machine that uses a large screw to pull a stream containing solids along a horizontal screen tube. Movement of the solids can be impeded by a weighted plate at the end of the tube. The pressure of this plate on the solid plug forces liquid out of the solids and through the holes in the sides of the screen tube and then along the effluent pipe. The screw will then push the remaining solids past the plate where they fall out onto a collection pad below.

5.3. Liquefaction Methods

The liquefaction methods of the disclosure generally entail subjecting slurries containing biomass solids to one or more hydrolyzing proteins, and/or forming biomass slurries in the presence of one or more hydrolyzing proteins, in order to reduce slurry viscosity or form a slurry with reduced viscosity, for example as a preparation step for a saccharification reaction.

The liquefaction method typically comprise incubating or forming a reaction mixture comprising a biomass slurry containing at least 5%, at least 8%, at least 10%, at least 12% or at least 14% by weight of pretreated biomass solids and an aqueous phase (e.g., water and/or hemicellulose hydrolysate) with one or more hydrolyzing protein (e.g., cellulases) for a period of several minutes to several hours. The biomass slurry solids content can be 15% by weight or greater, for example 16%, 18%, 20%, 22%, 24%, 26%, 28%, 30%, 32%, 34%, 36%, 38%, or 40%, by weight, and is usually is no more than 45% by weight. In some embodiments biomass slurry solids is 25% or less by weight. In specific embodiments, the biomass slurry solids content by weight is in a range bounded by any two of the foregoing embodiments, such as, but not limited to, from 5% to 25%, from 8% to 20%, from 10% to 22%, from 12% to 24%, from 14% to 24%, from 16% to 24%, from 16% to 22%, from 18% to 22%, from 16% to 30%, from 14% to 34%, from 14% to 28%, from 18% to 30%, from 18% to 40%, from 16% to 45%, etc.

The liquefaction reaction may be carried out at temperatures greater than room temperature, for example 30° C., 35° C., 40° C., 45° C., 50° C., 55° C., 56° C., 57° C., 58° C., 59° C., 60° C., 61° C., 62° C., 63° C., 64° C., 65° C., 66° C., 67° C., 68° C., 69° C., 70° C., 75° C., or 80° C. If minimizing saccharification and/or growth of contaminating microorganisms is desired, liquefaction can be carried out at 62° C. or greater. In specific embodiments, the temperature is in a range bounded by any two of the foregoing embodiments, such as, but not limited to, from 40° C. to 80° C., from 50° C. to 80° C., from 50° C. to 75° C., from 60° C. to 80° C., from 62° C. to 75° C., from 62° C. to 72° C., from 62° C. to 70° C., from 65° C. to 80° C., from 60° C. to 75° C., or from 65° C. to 75° C.

The liquefaction reaction is typically carried out for a period of time ranging from 2 minutes to 4 hours, more typically from 5 minutes and 3 hours, and yet more typically from 15 minutes to 1.5 or 2 hours. In specific embodiments, the liquefaction is carried out for a period of time ranging from 5 minutes to 2 hours, from 5 minutes to 1.5 hours, from 5 minutes to 1 hour, from 5 minutes to 0.5 hours, from 10 minutes to 2 hours, from 10 minutes to 1.5 hours, from 10 minutes to 1 hour, from 10 minutes to 0.5 hours, from 15 minutes to 2 hours, from 15 minutes to 1.5 hours, from 15 minutes to 1 hour, from 15 minutes to 0.5 hours, from 0.5 hour to 2 hours, from 0.5 hours to 1.5 hours, or from 0.5 hours to 1 hour.

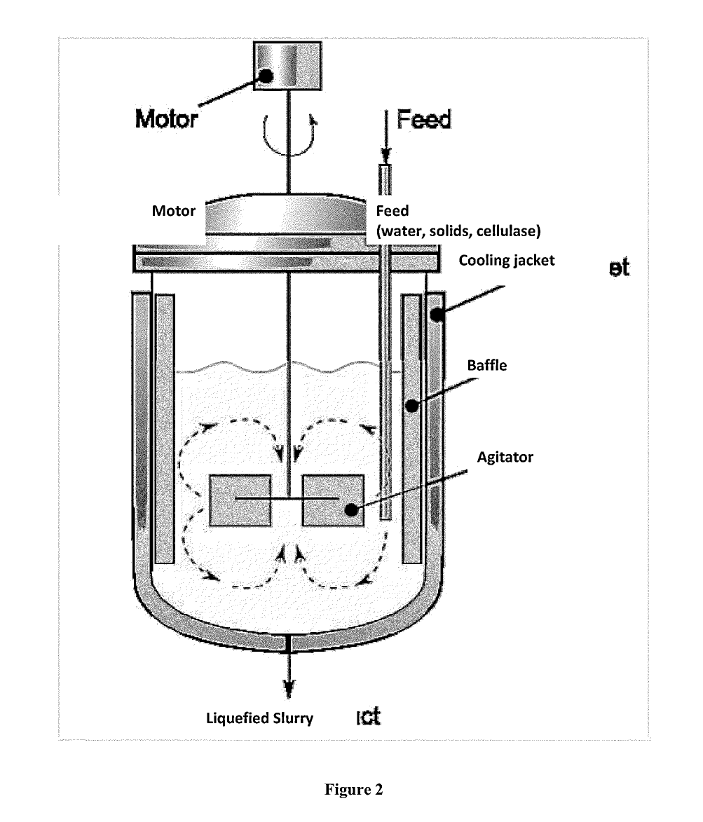

The liquefaction reaction can be performed in any suitable vessel, such as a batch reactor or a continuous reactor (e.g., a continuous stirred tank reactor (“CSTR”) as schematized in FIG. 2). The suitable vessel can be equipped with a means, such as impellers, for agitating the slurry. Reactor design is discussed in Lin, K.-H., and Van Ness, H. C. (in Perry, R. H. and Chilton, C. H. (eds), Chemical Engineer's Handbook, 5th Edition (1973) Chapter 4, McGraw-Hill, NY). The liquefaction reaction can be carried out as a batch process, or as a continuous process. Exemplary batch and continuous processes are described below.

5.3.1. Batch Mode

The liquefaction processes of the disclosure can be carried out in a batch mode. The methods typically entail batch combining a (1) biomass solids, (2) an aqueous phase; and (3) one or more cellulases in a reactor. The biomass solids, the aqueous phase, and one or more cellulases can be fed into the reactor together or separately. The reactor is emptied after a desired viscosity is reached, and another batch of (1) biomass solids, (2) an aqueous phase; and (3) one or more cellulases added to the reactor.

Any type of reactor can be used for batch mode liquefaction, which simply involves adding material, carrying out the liquefaction, reaction and then removing the liquefied material from the reactor.

Batch mode liquefactions are typically carried out for a period of time ranging from 2 minutes to 4 hours, more typically from 5 minutes and 3 hours, and yet more typically from 15 minutes to 1.5 or 2 hours. In specific embodiments, a batch mode liquefaction is carried out for a period of time ranging from 5 minutes to 2 hours, from 5 minutes to 1.5 hours, from 5 minutes to 1 hour, from 5 minutes to 0.5 hours, from 10 minutes to 2 hours, from 10 minutes to 1.5 hours, from 10 minutes to 1 hour, from 10 minutes to 0.5 hours, from 15 minutes to 2 hours, from 15 minutes to 1.5 hours, from 15 minutes to 1 hour, from 15 minutes to 0.5 hours, from 0.5 hour to 2 hours, from 0.5 hours to 1.5 hours, or from 0.5 hours to 1 hour.

5.3.2. Continuous Mode

The liquefaction processes of the disclosure advantageously reduces the need to stop and clean reactors and accordingly can be carried out in continuous mode, e.g., for periods of several days or longer (e.g., a week or more). The methods typically entail continuously feeding a reactor a (1) biomass solids, (2) an aqueous phase; and (3) one or more cellulases. The biomass solids, the aqueous phase, and one or more cellulases can be fed together or separately. After a slurry of a desired viscosity is generated, slurry is removed and additional components are added to the reactor at rates that maintains the volume and viscosity of slurry in the tank.

For liquefaction in continuous mode, any reactor can be used that allows equal input and output rates, e.g., a continuous stirred tank reactor, so that a steady state is achieved in the reactor and the fill level of the reactor remains constant.

In continuous mode, a liquefaction reaction is preferably carried out for a period of time of at least 12 hours or at least 18 hours, and up to up to 24 hours, up to 36 hours, up to 48 hours, up to 72 hours, up to 96 hours, up to 1 week or even more (e.g., up to 10 days, up to 2 weeks). In some embodiments, the reactions proceed for periods of at least one day, at least 2 days, at least 3 days, at least 4 days, at least 5 days, at least 6 days, at least a week, weeks, at least a month, months, at least a year, years or more. In some embodiments, reaction proceeds continuously without stopping. In continuous mode, the retention or residence time in the liquefaction vessel is preferably 2 hours or less (e.g., a time period of 0.25, 0.5, 1, 2, 5, 10, 15, 20, 25, 26, 27, 28, 29, or 30 minutes or 0.5, 0.75, 1, 1.5 or 2 hours, or for a time period ranging between any two of the foregoing values. The residence time in the liquefaction vessel typically ranges from 2 minutes to 4 hours, more typically from 5 minutes and 3 hours, and yet more typically from 15 minutes to 1.5 or 2 hours. In specific embodiments, the residence time ranges from 5 minutes to 2 hours, from 5 minutes to 1.5 hours, from 5 minutes to 1 hour, from 5 minutes to 0.5 hours, from 10 minutes to 2 hours, from 10 minutes to 1.5 hours, from 10 minutes to 1 hour, from 10 minutes to 0.5 hours, from 15 minutes to 2 hours, from 15 minutes to 1.5 hours, from 15 minutes to 1 hour, from 15 minutes to 0.5 hours, from 0.5 hour to 2 hours, from 0.5 hours to 1.5 hours, or from 0.5 hours to 1 hour.

5.3.3. Semi Continuous Mode

The liquefaction processes of the disclosure can be carried out in semicontinuous mode. Semicontinuous reactors, which have unequal input and output streams that eventually require the system to be reset to the starting condition, can be used.

5.4. Hydrolyzing Proteins

Hydrolyzing proteins suitable for practicing the liquefaction methods of the disclosure include cellulases, hemicellulases (including but not limited to xylanases, mannanases, beta-xylosidases), and other proteins that enhance saccharification by cellulase or hemicellulases, such carbohydrate esterases (including but not limited to acetyl xylan esterases and ferulic acid esterases), laccases (which are believed to act on lignin), and non-enzymatic proteins such as swollenins (which are thought to swell the cellulose (non-catalytically and make it more accessible to cellulases). As used herein, the term hydrolyzing proteins refers to a single protein, preferably an enzyme (yet more preferably a cellulase or hemicellulase) or a cocktail of different proteins, including one or more enzymes (preferably a cellulase and/or hemicellulase) and optionally one or more non-enzymatic proteins such as swollenins. The hydrolyzing proteins can have naturally occurring or engineered polypeptide sequences.

Biomass typically contains cellulose, which is hydrolyzable into glucose, cellobiose, and higher glucose polymers and includes dimers and oliogmers. Cellulose is hydrolysed into glucose by the carbohydrolytic cellulases. Thus the carbohydrolytic cellulases are examples of catalysts for the hydrolysis of cellulose. The prevalent understanding of the cellulolytic system divides the cellulases into three classes; exo-1,4-β-D-glucanases or cellobiohydrolases (CBH) (EC 3.2.1.91), which cleave off cellobiose units from the ends of cellulose chains; endo-1,4-β-D-glucanases (EG) (EC 3.2.1.4), which hydrolyse internal β-1,4-glucosidic bonds randomly in the cellulose chain; 1,4-β-D-glucosidase (EC 3.2.1.21), which hydrolyses cellobiose to glucose and also cleaves off glucose units from cellooligosaccharides. Therefore, if the biomass contains cellulose, suitable hydrolyzing enzymes include one or more cellulases.

Many biomasses include hemicellulose, which is hydrolyzable into xylan, glucuronoxylan, arabinoxylan, glucomannan, and xyloglucan. The different sugars in hemicellulose are liberated by the hemicellulases. The hemicellulytic system is more complex than the cellulolytic system due to the heterologous nature of hemicellulose. The systems may involve among others, endo-1,4-β-D-xylanases (EC 3.2.1.8), which hydrolyse internal bonds in the xylan chain; 1,4-β-D-xylosidases (EC 3.2.1.37), which attack xylooligosaccharides from the non-reducing end and liberate xylose; endo-1,4-β-D-mannanases (EC 3.2.1.78), which cleave internal bonds; 1,4-β-D-mannosidases (EC 3.2.1.25), which cleave mannooligosaccharides to mannose. The side groups are removed by a number of enzymes; such as α-D-galactosidases (EC 3.2.1.22), α-L-arabinofuranosidases (EC 3.2.1.55), α-D-glucuronidases (EC 3.2.1.139), cinnamoyl esterases (EC 3.1.1.), acetyl xylan esterases (EC 3.1.1.6) and feruloyl esterases (EC 3.1.1.73). Therefore, if the biomass contains hemicellulose, suitable hydrolyzing enzymes include one or more hemicellulases.

The cellulase cocktails suitable for practicing the liquefaction methods of the disclosure typically include one or more cellobiohydrolases, endoglucanases and/or β-glucosidases. Cellulase cocktails are compositions comprising two or more cellulases. In their crudest form, cellulase cocktails contain the microorganism culture that produced the enzyme components. “Cellulase cocktails” also refers to a crude fermentation product of the microorganisms. A crude fermentation is preferably a fermentation broth that has been separated from the microorganism cells and/or cellular debris (e.g., by centrifugation and/or filtration). In some cases, the enzymes in the broth can be optionally diluted, concentrated, partially purified or purified and/or dried.

Suitable cellulases include those of bacterial or fungal origin. Suitable cellulases include cellulases from the genera Bacillus, Pseudomonas, Trichoderma, Aspergillus, Chrysosporiuim, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259. The Trichoderma reesei cellulases are disclosed in U.S. Pat. No. 4,689,297, U.S. Pat. No. 5,814,501, U.S. Pat. No. 5,324,649, WO 92/06221 and WO 92/06165. Bacillus cellulases are disclosed in U.S. Pat. No. 6,562,612.

Commercially available cellulases or cellulase cocktails that can suitably be used in the present methods include, for example, CELLIC CTec (Novozymes), ACCELLERASE (Genencor), SPEZYME CP (Genencor), 22 CG (Novozymes), Biocellulase W (Kerry) and PYROLASE (Verenium).

In some embodiments, the cellulase cocktail includes one or more proteins not normally produced by the cellulase-producing microorganism. The non-native proteins can be foreign or engineered proteins recombinantly co-expressed with other cellulase cocktail components by a cellulase-producing microorganism (e.g., bacterium or fungus), or natively or recombinantly produced separately from other cellulase components (e.g., in a bacterium, plant or fungus) and added to the cellulase cocktail. Mixtures of enzymes from different organisms can also be used.

For liquefaction at high temperatures, thermostable cellulases can be used. Thermostable cellulases are known in the art and are also available commercially. See U.S. Pat. Nos. 7,510,857, 6,812,018 and 5,677,151; International Publication Nos. WO 1993/015186, WO 2010/048522, WO 1991/005039 and WO 2008/025164; European Publication Nos. EP 0885955 B1 and EP 2013338 A1; Jang & Chen, 2003, World Journal of Microbiology and Biotechnology 19(3):263-268; Heinzelman et al., 2009, Proc. Nat'l. Acad. Sci. U.S.A. 106(14):5451-5452; Rastogi et al., 2010. Bioresour Technol. 101(22):8798-806. Thermostable cellulases that can withstand higher temperatures are also available commercially, for example PYROLASE (from Verenium) and product nos. G8798 (a thermostable β-glucosidase); G8673 and G8548 (which are thermostable β-glucanases); C9499 and C9624 (which are thermostable cellulases from Clostridium thermocellum and Dictyoglomus turgidum, respectively) from Sigma Aldrich.

Hydrolyzing proteins can be used singly or in enzyme/cocktail blends in doses ranging from 5 μg to 20 mg protein per gram dry weight of solids in the slurry (e.g., 5 μg, 10 μg, 20 μg, 50 μg, 100 μg, 250 μg, 500 μg, 1 mg, 2 mg, 5 mg, 10 mg, or 20 mg protein per gram dry weight of solids in the slurry. In various embodiments, the dosage per gram dry weight of solids in the slurry is in a range bounded by any two of the foregoing embodiments, such as 10 μg to 250 μg, from 20 μg to 500 μg, from 50 μg to 250 μg, from 10 μg to 100 μg, or from 20 μg to 250 μg, from 100 μg to 10 mg, from 250 μg to 20 mg, etc. In specific embodiments, the hydrolyzing protein is an endoglucanase or an enzyme/cocktail blend in which at least 5%, at least 10%, at least 15%, at least 20%, at least 25%, at least 30%, at least 40% or at least 50% of the protein weight is composed of one or more endoglucanases.

Cellulases are preferably used in at doses ranging from 10 CTU to 500 CTU cellulase per gram dry weight of solids in the slurry (e.g., 10 CTU, 20 CTU, 30 CTU, 40 CTU, 50 CTU, 60 CTU, 80 CTU, 100 CTU, 125 CTU, 150 CTU, 175 CTU, 200 CTU, 250 CTU, 300 CTU, 400 CTU or 500 CTU). In various embodiments, the amount of cellulase per gram dry weight of solids in the slurry is in a range bounded by any two of the foregoing embodiments, such as 10 CTU to 200 CTU, from 20 CTU to 400 CTU, from 40 CTU to 250 CTU, from 10 CTU to 100 CTU, or from 20 CTU to 250 CTU, etc.

The term CTU as used herein refers to units of cellulase activity as measured using CELLAZYME T tablets (Megazyme, Co. Wickow, Ireland). The substrate in this assay is azurine-crosslinked Tamarind Xyloglucan (AZCL-Xyloglucan). This substrate is prepared by dyeing and cross-linking highly purified xyloglucan to produce a material which hydrates in water but is water insoluble. Hydrolysis by cellulase, for example, endo-(1-4)-b-D-glucanase, produces water soluble dyed fragments and the rate of release of these (increase in absorbance at 590 nm) can be related directly to enzyme activity. One CTU is defined as the amount of enzyme required to release one micromole of glucose reducing sugar-equivalents per minute from barley β-glucan (10 mg/ml) at pH 4.5 and 40° C. 7.5 CTUs of cellulase cocktail corresponds to approximately 1 filter paper unit (“FPU”). As used herein, the term FPU refers to filter paper units as determined by the method of Adney and Baker, Laboratory Analytical Procedure #006 (“LAP-006”), “Measurement of cellulase activity,” Aug. 12, 1996, the USA National Renewable Energy Laboratory (NREL), which is expressly incorporated by reference herein in entirety. 1 mg of total protein of a T. reesei cellulase cocktail (as measured by the Bradford assay) corresponds to approximately 27.4 CTU. In alternative embodiments of the liquefaction methods of the disclosure, the reference to enzyme dosages in “CTUs” can be replaced with the approximate corresponding amounts of enzyme by protein mass or FPUs, using the conversion of 36.5 μg of a cellulase or cellulase cocktail or 0.133 FPU of a cellulase or cellulase cocktail per CTU. Accordingly, in these alternative embodiments, enzyme dosages referred to by CTUs in the various aspects of the disclosure are substituted by the corresponding dosage in protein mass or FPU. Thus, for example, alternatives to an embodiment of the liquefaction methods in which the enzyme dose is 20 to 400 CTU are embodiments in which the enzyme dose is 730 μg to 14.6 mg protein or a cellulase or cellulase cocktail characterized by an activity of 2.67 to 53.33 FPU.

5.5. Saccharification of Liquefied Biomass

The liquefied biomass produced in accordance with methods disclosed herein can suitably be used in saccharification reactions to produce simple sugars for fermentation or chemical syntheses. Accordingly, the present disclosure provides methods for saccharification comprising contacting liquefied biomass with hydrolyzing enzymes and, optionally, subjecting the resulting sugars to fermentation by a microorganism. The saccharification can take place in the reactor in which the liquefaction step was carried out, or more preferably the liquefied biomass is transferred (e.g., pumped) into a different reactor for saccharification.

The liquefied biomass slurry is then further hydrolyzed in the presence of saccharifying enzymes to release oligosaccharides and/or monosaccharides in a hydrolyzate. Saccharification enzymes and methods for biomass treatment are reviewed in Lynd et al., 2002, Microbiol. Mol. Biol. Rev. 66:506-577). Saccharification enzymes can include the cellulases and/or the hemicellulases described in Section 4.4. The enzymes can be purchased commercially or produced biologically by recombinant or non-recombinant microorganisms, which optionally includes production in a consolidated bioprocessing (CBP) process, which featuring cellulase production (e.g., by the fermenting microorganism), cellulose hydrolysis and fermentation in one step (see Lynd et al., 2005, Current Opinion in Biotechnology 16:577-583).

The saccharification can be performed batch-wise or as a continuous process. The saccharification can also be performed in one step, or in a number of steps. For example, different enzymes required for saccharification may exhibit different pH or temperature optima. A primary treatment can be performed with enzyme(s) at one temperature and pH, followed by secondary or tertiary (or more) treatments with different enzyme(s) at different temperatures and/or pH. In addition, treatment with different enzymes in sequential steps may be at the same pH and/or temperature, or different pHs and temperatures, such as using hemicellulases stable and more active at higher pHs and temperatures followed by cellulases that are active at lower pHs and temperatures.

The degree of solubilization of sugars from biomass following saccharification can be monitored by measuring the release of monosaccharides and oligosaccharides. Methods to measure monosaccharides and oligosaccharides are well known in the art. For example, the concentration of reducing sugars can be determined using the 1,3-dinitrosalicylic (DNS) acid assay (Miller, 1959, Anal. Chem. 31:426-428). Alternatively, sugars can be measured by HPLC using an appropriate column as is well known to the skilled artisan.

5.6. Uses of Saccharified Biomass

The saccharified biomass can be made into a number of bio-based products, via processes such as, e.g., microbial fermentation and/or chemical synthesis. As used herein, “microbial fermentation” refers to a process of growing and harvesting fermenting microorganisms under suitable conditions. The fermenting microorganism can be any microorganism suitable for use in a desired fermentation process for the production of bio-based products. Suitable fermenting microorganisms include, without limitation, filamentous fungi, yeast, and bacteria. The saccharified biomass can, for example, be made it into a fuel (e.g., a biofuel such as a bioethanol, biobutanol, biomethanol, a biopropanol, a biodiesel, a jet fuel, or the like) via fermentation and/or chemical synthesis. The saccharified biomass can, for example, also be made into a commodity chemical (e.g., ascorbic acid, isoprene, 1,3-propanediol), lipids, amino acids, polypeptides, and enzymes, via fermentation and/or chemical synthesis.

Thus, in certain aspects, liquefied biomass can be used in the generation of ethanol from biomass in either separate or simultaneous saccharification and fermentation processes. Separate saccharification and fermentation is a process whereby cellulose present in biomass is saccharified into simple sugars (e.g., glucose) and the simple sugars subsequently fermented by microorganisms (e.g., yeast) into ethanol. Simultaneous saccharification and fermentation (“SSF”) is a process whereby cellulose present in biomass is saccharified into simple sugars (e.g., glucose) and, at the same time and in the same reactor, microorganisms (e.g., yeast) ferment the simple sugars into ethanol. SSF can further include the step of cellulase production, in a process referred to as consolidation bioprocessing (“CBP”). CBP thus includes cellulase production, cellulose hydrolysis and fermentation in one step (see Lynd et al., 2005, Current Opinion in Biotechnology 16:577-583). The cellulase producer can be the fermenting microorganism.

The fermentation of sugars to ethanol may be carried out by one or more appropriate ethanologens in single or multistep fermentations. Ethanologens can be wild type microorganisms or recombinant microorganisms, and include Escherichia, Zymomonas, Saccharomyces, Candida, Pichia, Streptomyces, Bacillus, Lactobacillus, and Clostridium. Particularly suitable species of ethanologens include Escherichia coli, Zymomonas mobilis, Bacillus stearothermophilus, Saccharomyces cerevisiae, Clostridia thermocellum, Thermoanaerobacterium saccharolyticum, and Pichia stipitis. Genetically modified strains of E. coli or Zymomonas mobilis can be used for ethanol production (see, e.g., Underwood et al., 2002, Appl. Environ. Microbiol. 68:6263-6272 and US 2003/0162271 A1).

Fermentation of sugars to ethanol, acetone, and butanol (ABE fermentation) by solventogenic Clostridia is well known (Jones and Woods, 1986, Microbiol. Rev. 50:484-524). A fermentation process for producing butanol, acetone and ethanol, using a mutant strain of Clostridium acetobutylicum is described in U.S. Pat. No. 5,192,673. The use of a mutant strain of Clostridium beijerinckii to produce butanol, acetone, and ethanol is described in U.S. Pat. No. 6,358,717.

5.7. Recovery of Fermentation Products

Fermentation products can be recovered using various methods known in the art. Products may be separated from other fermentation components by centrifugation, filtration, microfiltration, and nanofiltration. Products may be extracted by ion exchange, solvent extraction, or electrodialysis. Flocculating agents can be used to aid in product separation. Solids may be removed from the fermentation medium by centrifugation, filtration, decantation, or the like.

After or during fermentation, the fermentation product, e.g., ethanol, can be separated from the fermentation broth by any of the many conventional techniques known to separate ethanol from aqueous solutions. These methods include evaporation, distillation, azeotropic distillation, solvent extraction, liquid-liquid extraction, membrane separation, membrane evaporation, adsorption, gas stripping, pervaporation, and the like. As a specific example, ethanol may be isolated from the fermentation medium using methods known in the art for ABE fermentations (see for example, Durre, 1998, Appl. Microbiol. Biotechnol. 49:639-648; Groot et al., 1992, Process. Biochem. 27:61-75; and references therein).

5.8. Waste Recycling

When recovering fermentation products, for example during distillation of ethanol, the fermented contents are then typically discharged as a slurry to the beer well (referred to as the “beer stream”) and from there to the beer still where the ethanol is removed by distillation. The remainder, after distillation, is known as the still bottoms or stillage, and consists of a large amount of water together with the spent solids. The stillage typically includes both liquid and solid material. The liquid and solid can be separated by, for example, centrifugation, which typically requires the addition of water to thin the stillage to a consistency suitable for centrifugation. Following centrifugation, the solids typically contain absorbed or adsorbed water as well as water in the interstitial spaces of the solids. This water is typically removed by drying the solids with thermal energy. The removal of water from process streams having a high water content is costly, energy intensive and time consuming.

The liquefaction methods of the disclosure provide additional environmental and economical benefits by reducing the amount of water required in the post-distillation processing of waste materials. In particular, use of liquefied biomass in the saccharification and fermentation processes results in stillage that requires the addition of less water (e.g., at least 30%, at least 40%, at least 50%, at least 60%, at least 70% less water) than would be added to achieve a consistency suitable for centrifugation when biomass not subject to liquefaction by hydrolyzing proteins, and can in some cases results in stillage that does not require the addition of any water prior to centrifugation.

### 6. EXAMPLES

### 6.1. Example 1

### Liquefaction of Wood Samples Using Cellulase

The ability of cellulase to liquefy three wood pulp samples (eucalyptus, mixed pine, and radiata pine) was tested. 0.25 grams pulp solids were added to a 10 mL reaction vial. Three different cellulase concentrations (0.5×, 1× and 2× enzyme (corresponding to 3 mg/ml, 6 mg/ml, 12 mg/ml total protein of an enzyme cocktail comprising CBH1, CBH2, EG and BG) and buffer were added to bring the final volume in each vial to 5 mL, 5% solids. The vials were shaken in a hybridization oven at 50° C. for 2 days to mix their contents, and the contents were sampled at predetermined intervals. The samples collected were analyzed via HPLC to determine their sugar composition and content.

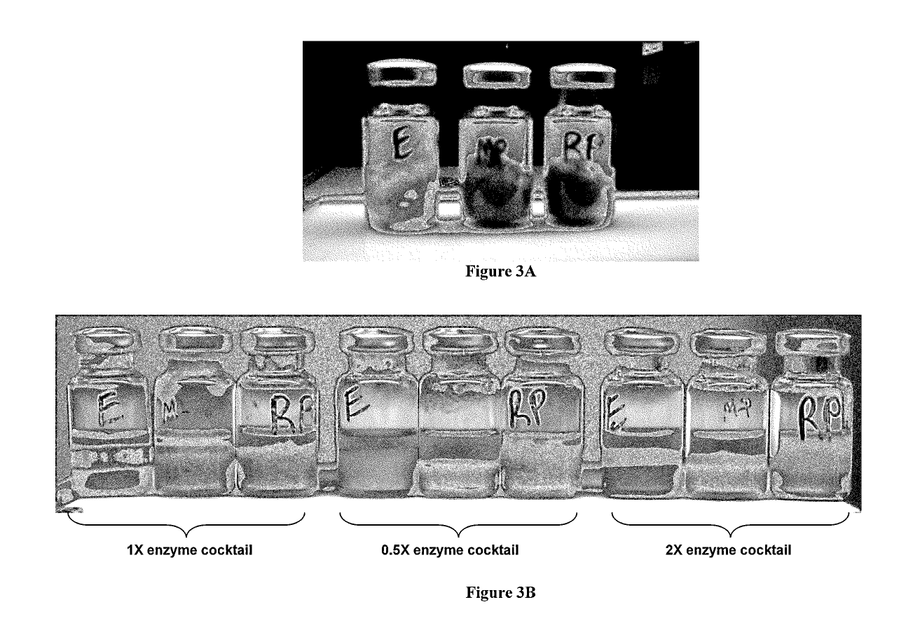

Once buffer was added to pulp control samples, all liquid was absorbed (FIG. 3A), and mixing in the vials was impeded by the viscosity of the samples. In control vials lacking cellulase, the vial contents maintained their viscous appearance after 2 days at 50° C. (data not shown). In the enzyme-treated samples, viscosity was reduced proportional to the enzyme dose (FIG. 3B). The degree of glucan saccharification in the different samples at different time points is summarized in Table 1.

This study suggests that pulp viscosity might retard saccharification reaction progress.

### 6.2. Example 2

### Monitoring of Agitator Motor Current as a Way to Measure “Flowability” or “Mixability” of Biomass Slurry

A method for monitoring motor current (i.e., energy introduced into a reactor) using an electronically filtered voltage output for the motor controller was implemented. Data loggers were integrated to provide continuous monitoring of current during fermentations.

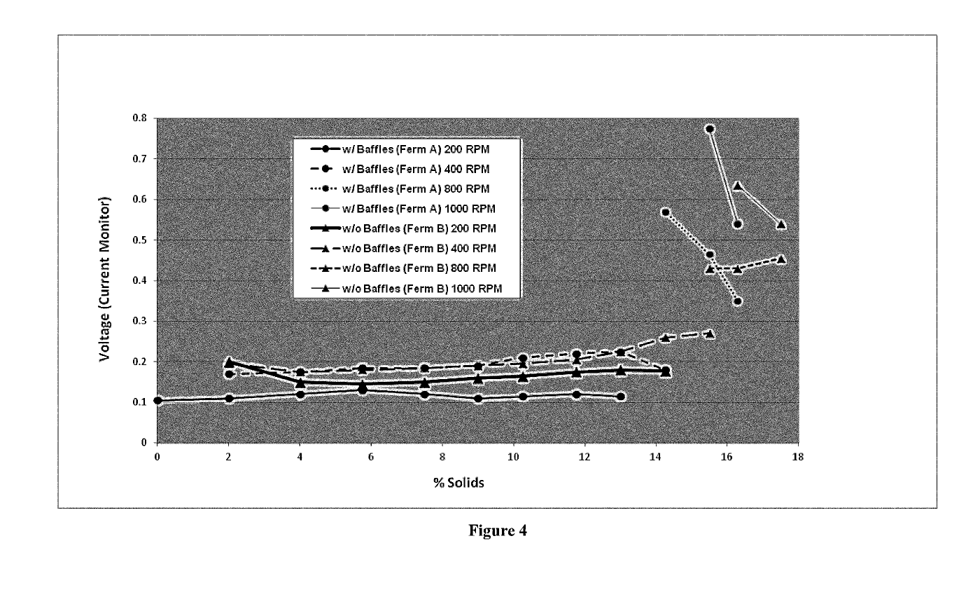

The flowability/viscosity profile of pretreated sugarcane bagasse during SSF (using Kerry Biocellulase W at pH5.6 and 35° C.) was monitored using motor current as a surrogate measure of viscosity. In a first study, motor current vs. % solids was measured. Motor current increased with increasing percentage solids up to about 15%. Beyond that level of solids, the material no longer mixes and the fermenter impeller spins without mixing and the motor current is actually reduced as would be characteristic for a pseudoplastic/thixotropic fluid (FIG. 4).

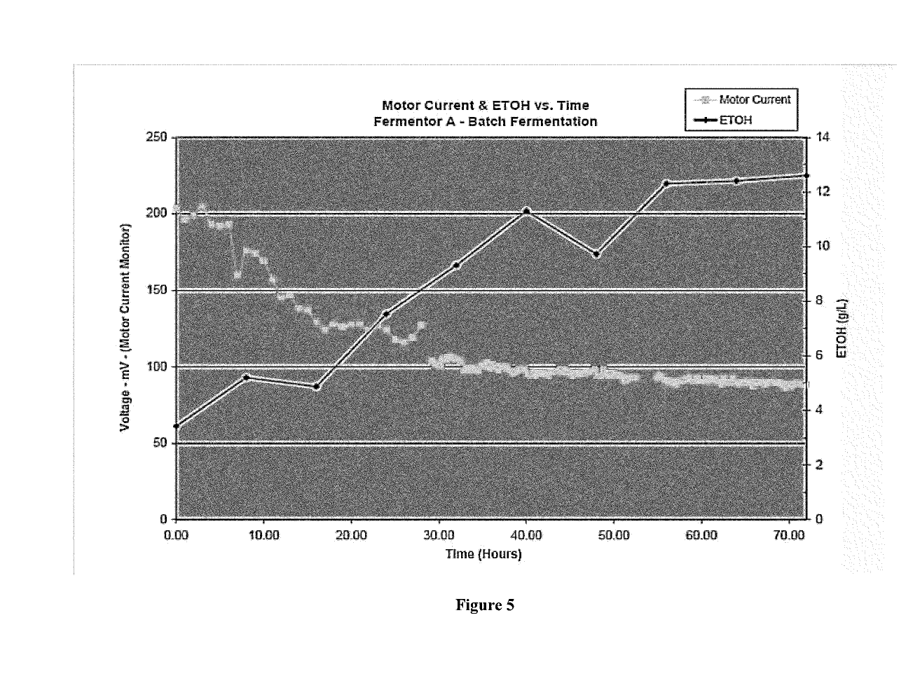

In a second study, a low solids (10%) SSF was performed with motor current monitoring. Data shows reduced motor current as the fermentation progresses (FIG. 5).

### 6.3. Example 3

### Liquefaction of Sugar Cane Bagasse (Run A)

6.3.1. Overview

Sugar cane bagasse which had been pretreated with sulfuric acid and neutralized with lime was analyzed for rheological properties when treated with Kerry Biocellulase W. This study demonstrates that early low dosing of the biomass with the cellulase results in dramatic improvements in the viscosity reduction of the biomass slurry. One set of conditions resulted in a 35-fold reduction in viscosity.

6.3.2. Materials and Methods

The initial solids content of the limed slurry was measured to be 11% solids, pH 5.22. Additional biomass was added to the 11% lime slurry to achieve a 14% solids content. The solids addition changed the pH to 5.02.

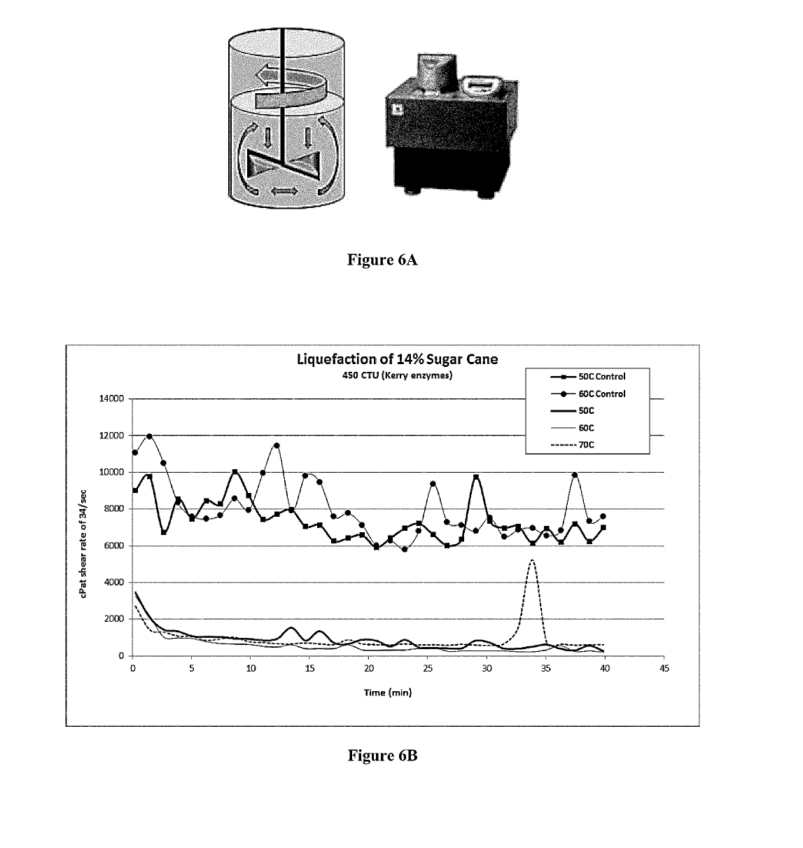

Thirty grams (˜30 mL) of 14% slurry was aliquoted into a RVA4 canister. The RVA has an internal heater that allows the analysis to be performed at a specified temperature (e.g., 50° C., 60° C., and 70° C.). The slurry aliquot was preheated for thirty minutes at the specified temperature. Enzyme was dosed into to the 14% slurry at 1.62 mL and then the sample was immediately placed in the RVA4 viscometer (Perten Instruments; illustrated in FIG. 6A) for a 40 minute analysis. The viscosity analysis was performed at a shear rate of 34/sec (100 rpm). The controls were performed by adding 1.62 mL water. Enzymatic dosing experiments were performed at 450, 300, and 150 CTU (corresponding to approximately 107, 71, and 35 CTU/gram solids, respectively).

6.3.3. Results

The controls (14% lime slurry+1.62 mL water) showed an initial viscosity of˜8,000-10,000 cP and thinned to 6,000-7,000 cP within the first 15 minutes and then remained steady for the remaining 25 minutes.

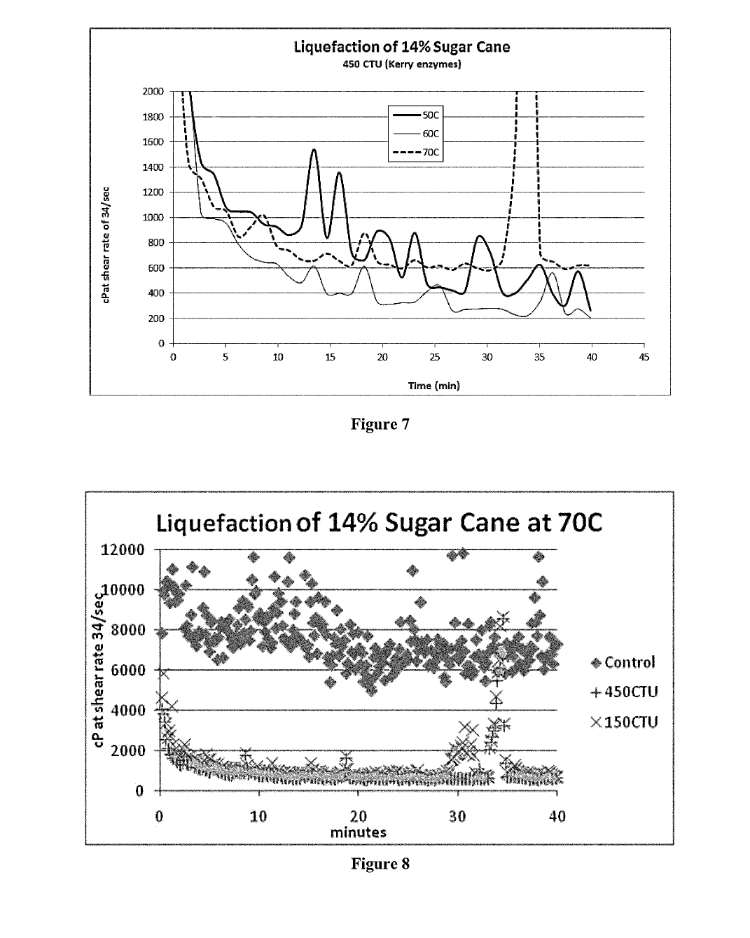

All enzymatically treated samples showed dramatic viscosity reduction during the first 10 minutes of the analysis, which continued up to 20 minutes (FIG. 6B); minimal viscosity reduction was observed past 20 minutes at 50° C. and 60° C. and no reduction was observed past that time interval for 70° C. treatment. At 60° C. (FIG. 7), the observed viscosity at this temperature after 40 minutes was 202 cP+12 cP at a shear rate of 34/sec; 50° C. treatment was ˜264 cP+27 cP and 70° C. treatment was ˜607 cP+155 cP.

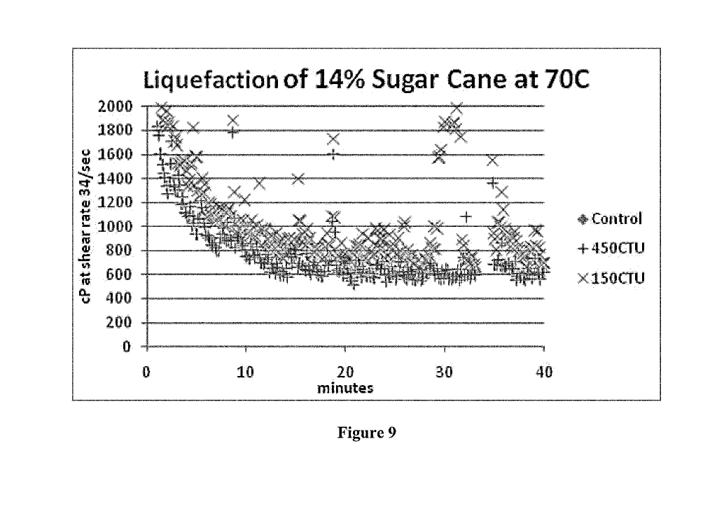

The best dosing for enzyme liquefaction in the 70° C. application study was observed to be 450 CTU (˜607 cP+155 cP) (FIG. 8). Significant reduction in viscosity was also observed at 150 CTU (˜696 cP+47 cP) (FIG. 9).

It was also possible to observe visual differences between the three dosing conditions. Slurries dosed with 450 CTU cellulase were more fluid and had fewer clumps than the slurries dosed with 150 CTU cellulase. All three enzyme-dosed samples showed a clear difference in fluidity relative to the untreated samples. Untreated 14% slurry did not appear to flow in a 50 mL conical tube, however, all enzyme treated samples displayed a flow when rocked back and forth.

### 6.4. Example 4

### Liquefaction of Sugar Cane Bagasse (Run B)

A study similar to that of Example 3 was carried out. Samples containing 14% slurry (18.44 g solids in 45 mL) were treated with 25, 50, 100 or 150 CTU of a cellulase cocktail per gram solids at 60° C. or 70° C. Most test samples were buffered to a pH of 4.5; one sample (at 60° C.) was unbuffered (with a pH of 2.5) and treated with enzymes; and no-enzyme control samples were unbuffered and had a pH of 2.5.

The resulting viscosities were determined as described in Example 3 and soluble sugar content was determined using HPLC. The concentration results as well as extent of cellulose saccharification (sum of cellobiose and glucose) and hemicellulose saccharification (xylose) are shown in Table 2.

The percentage of viscosity reduction over time is summarized in Tables 3 and 4 for the CTU/g samples at 60° C. and 70° C., respectively.

### 6.5. Example 5

### Liquefaction at Demonstration Plant Scale

6.5.1. Materials & Methods

Biomass was washed and dewatered and added at a constant rate to a hydrolyzer where it was pretreated with dilute sulfuric acid and raised temperature for several minutes. The hydrolyzed biomass was then explosively decompressed through a valve and accumulated in a slurry tank where it was slurried to 5% consistency with additional liquid. It was then pumped to a screw press where the slurry was dewatered to form a cake. The cake dropped out of the screw press into a mixer where it was combined with recycled liquefied slurry, lime (to adjust the pH), cellulase cocktail (to reduce the viscosity), and water and dropped into a continuously stirred and cycled 1600-gallon capacity liquefaction tank that is typically operated at a fill level of 500 to 1,000 gallons. The amount of water that was added at the mixer was reduced after the slurry in the tank began to thin. This increased the concentration of solids in the tank while maintaining the viscosity and pump load (current draw). A progressive cavity pump was used to circulate the slurry through a loop that went to the fermentation tanks and returned to the mixer where the slurry combined with fresh cake.

6.5.2. Results

When enzyme was added to the slurry and reduced the viscosity, the current required to drive the pump dropped. At this point, the water was reduced that was slurrying the cake and the consistency of the slurry increased (see FIG. 10A). Successful liquefaction is observed when the slope of the current vs. consistency is reduced. The current at which the pump ceases to propel slurry or the consistency at which the slurry ceases to flow represent the upper operational limits of the progressive cavity pump. When liquefaction takes place, the current required to propel the slurry decreases, and the consistency at which the slurry flows increases. The liquefaction tank operated in continuous mode where the maintained volume was a 50 minute retention time.

In a previous run that did not utilize cellulase, the current required to drive the pump was measured and plotted against the consistency of the slurry that was being pumped (FIG. 10B).

An analysis of the consistency in the liquefaction tank during the run indicated that a total solids consistency of 20.0% and an insoluble fiber ratio of 16.5% had been achieved where previously the consistency of the slurry had been limited operationally to ˜16% total solids consistency.

In addition to achieving higher consistency in the slurry tank, a higher consistency was achieved in the fermentation tank where saccharification and fermentation were conducted. The higher consistency meant that a higher concentration of cellulose was present. A higher cellulose concentration allows a higher sugar concentration to be achieved through saccharification and more ethanol to be produced in fermentation.

In addition to reducing the amount of water required to slurry the dewatered cake, several other advantages were realized. Post fermentation processes such as distillation of ethanol and inactivation of the fermentation reactions proceeded faster with fewer system stoppages due to fiber plugging the distillation column and less fiber accumulating in holding tanks prior to and following distillation. Post distillation separation of the liquid and fiber with a centrifuge also required no water to thin the slurry for centrifugation where previously a fresh water addition roughly equal to the stream going to centrifugation was required.

### 6.6. Example 6

### Formation of Slurry Containing Hemicelluloses

The process described in Example 5 utilized 3 screw presses in the solid liquid separation stage and counter current-flowing water to wash the hemicellulose hydrolysate from the cake. In a variation of this process a single screw press was used to dewater the cake and no washing was employed to remove the hemicellulose hydrolysate. Even in the presence of hemicellulose hydrolysate, which could theoretically inhibit the enzymes in the cellulase cocktail, there was clear evidence of liquefaction, which was reflected by an improved ability to pump the slurry and by a slurry consistency exceeding 16% (the limit previously established without enzyme addition).

### 6.7. Example 7

### Liquefaction of Pretreated Sorghum

Sugar Graze Ultra sorghum was harvested. Feedstock was washed to remove some of the organic acids and fed into the hydrolyzer. Hydrolysis conditions were ˜163° C., 1.1% sulfuric acid, and 15 minute retention time at a 3:1 liquid-to-solid ratio (LSR), followed by steam explosion. The material was passed through one screw press to obtain a cake at 33.0% insoluble solids. Compositional analysis determined a residual glucan content of 55.4% and residual xylan of 2.8%.

This material was subject to viscosity testing. Measurements were taken using a DV-E-HB Brookfield vane viscometer at pH5.4, 50° C. Pretreated biomass was weighed into a 600 mL beaker and mixed with 50 mM NaCitrate pH5.5 buffer to create a slurry at 18% solids. The pH was adjusted to 5.4 using sulfuric acid and ammonia, and the slurry preheated to 50° C. The appropriate volume of concentrated enzyme stock (Biocellulase W, Kerry Biosciences) was added to the biomass slurry to achieve 25 CTU/gram solids. The slurry was then stirred briefly prior to starting measurement on the viscometer. For each timepoint measurement, the viscometer vane was lowered into the biomass slurry and the initial viscosity (at least the first 1.5 revolutions) measured, followed by the next three viscosity measurements, which were recorded and averaged (steady-state viscosity measure). This measurement process was repeated at time points between 20 seconds and 30 minutes, with multiple measurements taken in the first 5 minutes to capture initial reaction rates. This procedure was used to measure viscosity changes at multiple rotational speeds between 2 rpm and 100 rpm.

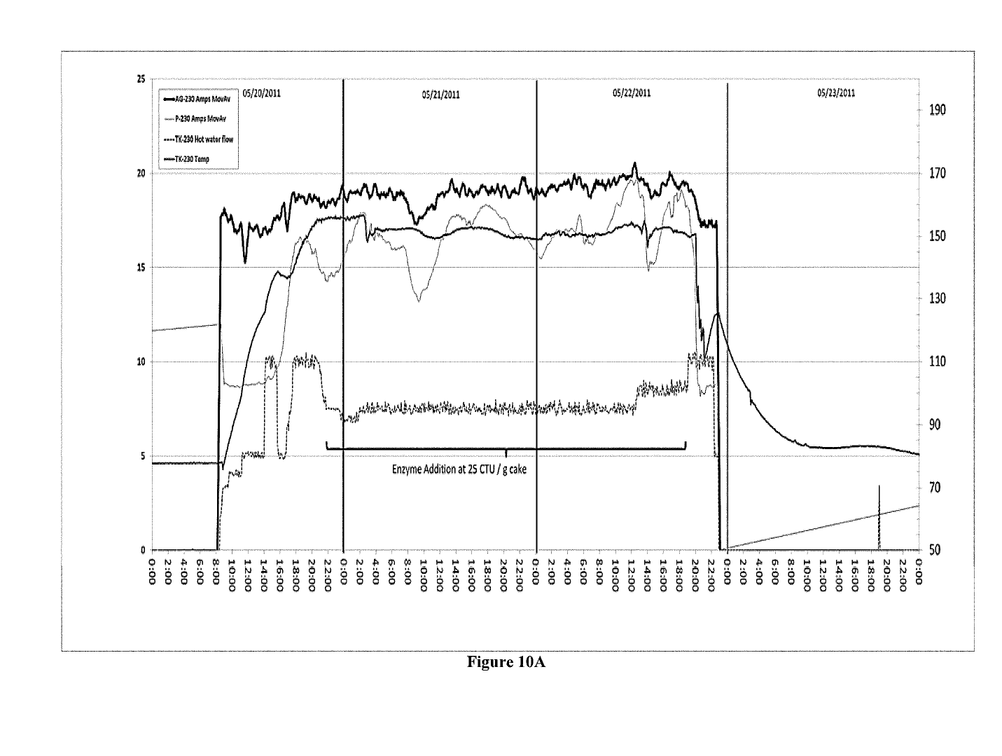

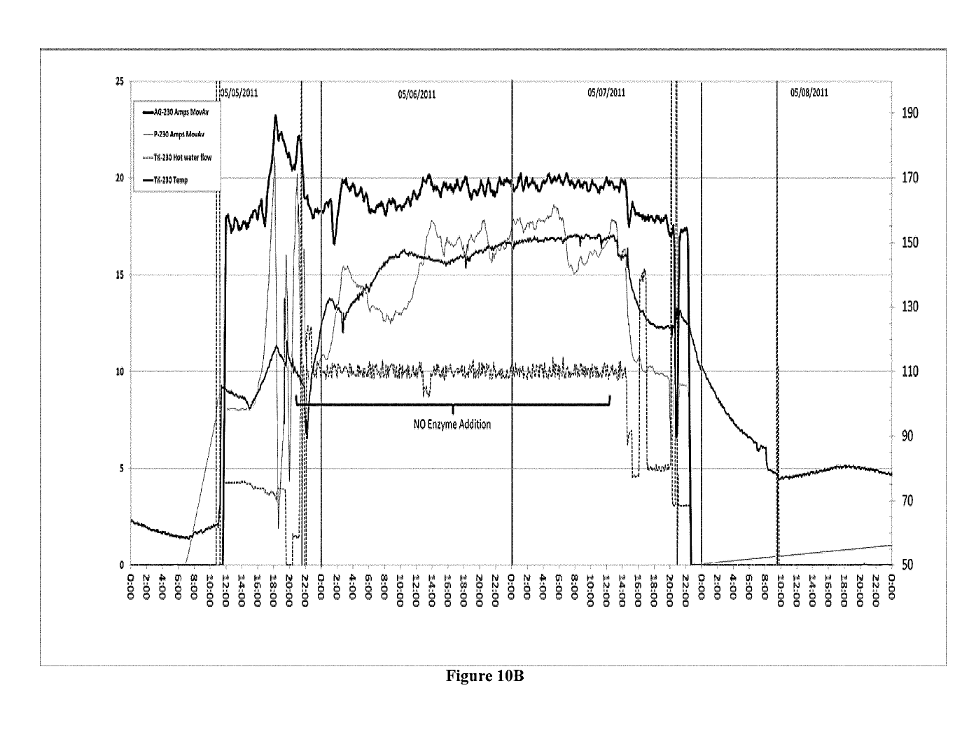

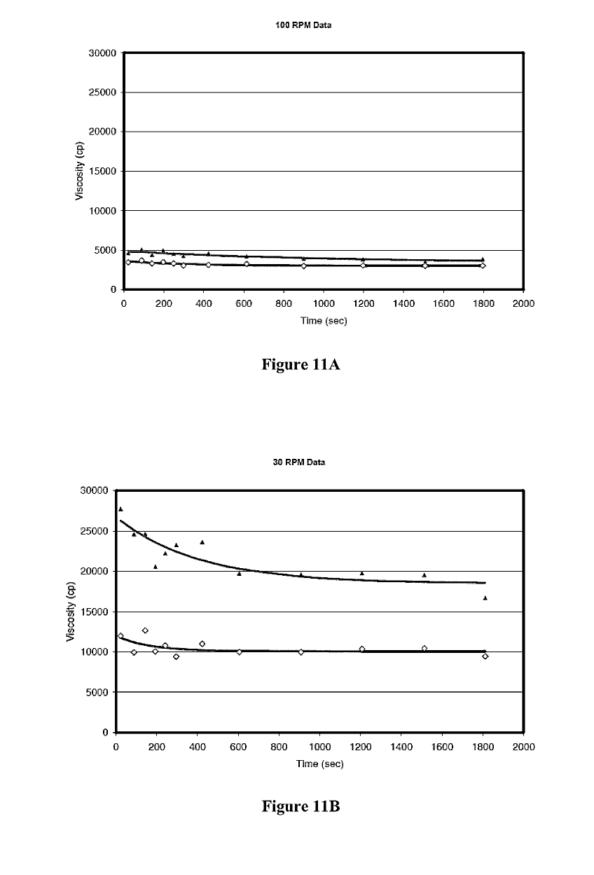

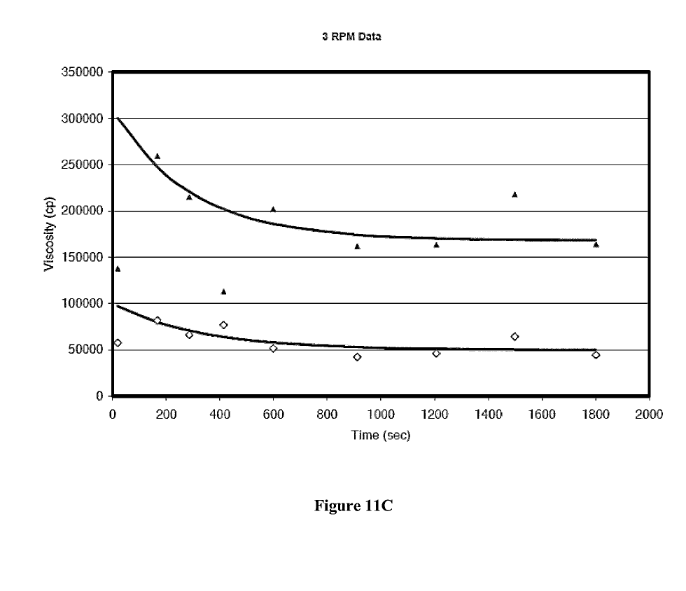

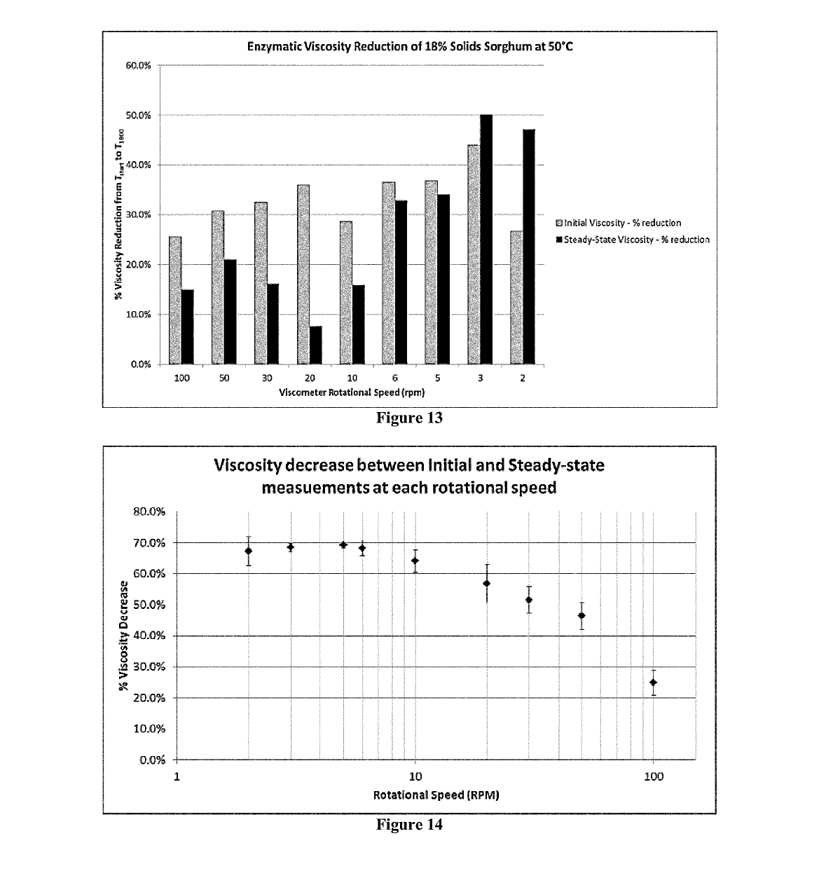

Enzymatic viscosity reduction was observed over the 30 minute liquefaction (FIGS. 11-13). Table 5 shows the viscosity measurements at selected time points. The effect appears to happen primarily within the first 900 seconds, following a first order exponential function where the initial viscosity decreases and levels off at a constant value after a period of time. As seen in FIG. 13, the magnitude of viscosity reduction varied between the initial measurement (25-44% viscosity reduction) and steady-state measurements (7.5-50% viscosity reduction), as well as the different rotational speeds.

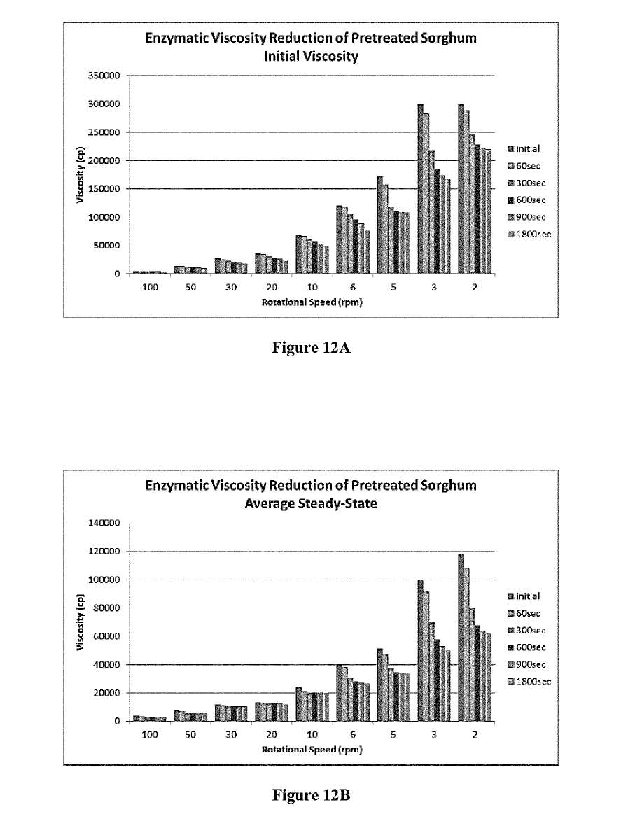

The initial viscosity measurements at each time point (FIG. 12A) were dramatically higher than the averaged steady-state readings (FIG. 12B). FIG. 14 shows the magnitude of this decrease in viscosity, averaged across six time points, for each rotational speed. The primary cause of this rapid decrease can likely be attributed to the shear-thinning behavior of the slurry. This reduction is highest for speeds less than 10 rpm, with the steady-state viscosity ˜70% lower than the initial viscosity. Above 10 rpm there is a decline in the magnitude of shear-thinning observed. At 100 rpm rotational speed the steady-state viscosity is approximately 25% lower than the initial measurement.

### 6.8. Example 8

### Liquefaction of Pretreated Napier Grass

6.8.1. Hydrolysis/Steam-Explosion

Napier grass was harvested from Highlands, Fla. pretreated at a variety of conditions to compare the effects of hydrolysis acid and steam explosion on the viscosity of biomass slurries, and on enzymatic liquefaction of these slurries.

The feedstock was well washed to remove organic acids and pre-steamed at 100° C. for 15 minutes. 2000 OD g of material was weighed into the reactor and impregnated with acid solution at a 10:1 liquid-to-solid ratio (LSR) for the specified retention time and temperature (Table 6). Acid concentrations were determined by targeting equivalent normality to the 0.5% sulfuric acid baseline condition. Hydrolysis reactions were run in duplicate. After hydrolysis the reactor was drained and the resulting cake was pressed to a target consistency of 33%, 2:1 LSR. Aliquots of this unexploded pressed material were collected for further analysis, while the remaining material was placed in the steam-explosion reactor. Steam explosion involved a 2-minute ramp to temperature, holding at temperature for 2.5 minutes, and then steam exploding by rapid release of the pressure. Approximately 3 OD kg of steam exploded material were collected for each condition for further analysis

6.8.2. Compositional Analysis

Compositional analysis was performed on the resulting unexploded and steam-exploded cakes using protocols adapted from NREL standard LAPs. The glucan, xylan, and insoluble solids content are listed in Table 7.

6.8.3. Physical Appearance

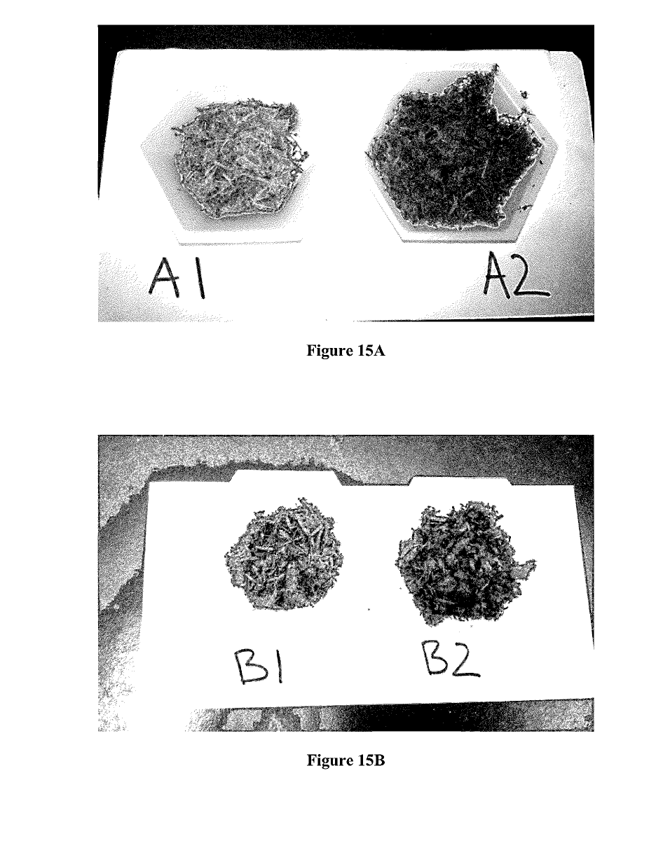

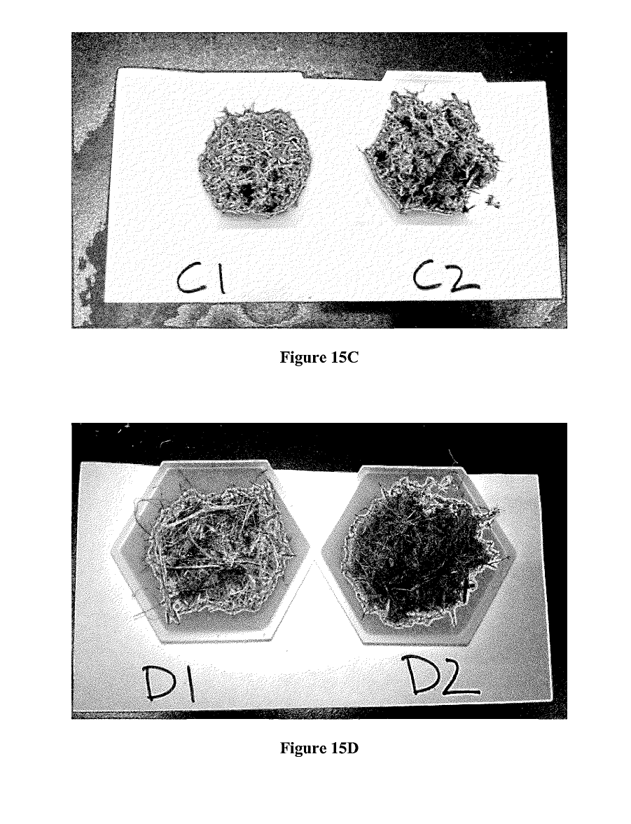

Hydrolysis conditions had a significant impact on the appearance of the pretreated cakes (FIG. 15). Samples B1 and B2 (FIG. 15B), pretreated with nitric acid, had the smallest particle size and appeared to be the most homogeneous of all the samples. Even though the percent insoluble solids were similar to the other acid pretreatments (Table 7), these materials appeared to be wetter than the others. These were also the darkest in color and resembled potting soil. The sulfuric acid-treated samples, A1 and A2 (FIG. 15A), appeared to have slightly larger average fiber length compared to the B samples, but were still fairly homogeneous. The C, D, and E samples had progressively larger fiber lengths, with some fibers longer than 5 or 6 inches remaining, even after steam-explosion (FIGS. 15C-E). These materials appeared to be much drier than the A and B cakes, even though the percent insoluble solids were similar. The apparent capacity to absorb moisture and the long fiber lengths, especially for the phosphoric and autohydrolysis samples, lead to a significant amount of tangling and clumping of the fibers, which made it challenging to work with in small scale tests and lead to some variability in the viscosity measurements. Qualitatively, fiber homogeneity appeared to be increased after steam explosion in all cakes, but remained relatively low in the D and E samples. Fiber analysis was performed to assess this more quantitatively.

6.8.4. Fiber Analysis

The pretreated cakes were analyzed by Bauer-McNett fiber classification, which involved passing a slurry containing ˜10 OD g of biomass through a series of five tanks fitted with screens of decreasing pore sizes. This separates the material into five distinct size classes. Water flows into the first tank, which fills and cascades into the next tank and so on. Each tank has a stirrer to facilitate movement of material across the screens. Tests were run for 20 minutes, and after each test was completed the tanks were drained and the biomass filtered through muslin, then dried and weighed.

The percent of biomass collected in each size class is shown in Table 8. Nitric acid pretreatment resulted in a higher proportion of the biomass in the smaller size classes, with only about 30% retained on the largest screen. Sulfuric, phosphoric, and acetic acid hydrolysis resulted in increasingly larger fibers, while autohydrolysis produced the highest proportion of material (˜70%) retained in the largest size class. Steam explosion did not significantly affect the distribution of materials in each size class within an acid treatment condition.

The cakes were also subjected to Morfi fiber analysis, which is an automated image analysis system that provides data on fiber size and shape, including length, width, and fines. The slurry was diluted to ˜50 mg/L, large pieces were removed (they are not measured by this method and could plug up the cell), and the dilute slurry passed through the cell for image analysis. Morfi data are reported in Table 9 for the ten pretreatment conditions.

### 6.9. Example 9

### Viscosity Analysis

The ten pretreated cakes described in Table 6 were subject to viscosity testing. Measurements were taken using a DV-E-HB Brookfield vane viscometer at pH5.5, 60° C. Pretreated biomass was weighed into a beaker and mixed with 50 mM NaCitrate pH5.5 buffer to the target % solids+2% (e.g., for a final 10% solids reaction, this initial slurry was prepared at 12%). Materials were prepared to act as solutions rather than wet bulk material. The pH was adjusted to 5.5 using sulfuric acid or ammonia, and the slurry preheated to 60° C. The appropriate volume of enzyme stock was diluted in preheated citrate buffer and the enzyme/buffer solution was added to the biomass slurry bring the reaction to the target consistency with an enzyme dose of 25 CTU TR1/g solids. The slurry was stirred briefly prior to initiating measurement on the viscometer. For each time point measurement, the vane was lowered into the biomass slurry and the initial viscosity (at least the first 1.5 revolutions) measured, followed by the next three viscosity measurements, which were recorded and averaged. This measurement process was repeated at time points between 20 seconds and 30 minutes (to ˜100 minutes for samples E1 and E2), with multiple measurements taken in the first 5 minutes to capture initial reaction rates. Appropriate rotational speeds were selected to obtain reliable viscosity data for the hydrolyzed material. Shear-thinning and enzymatic time constants were calculated, as well as percent viscosity reduction for each experiment.

6.9.1. Viscosity Results

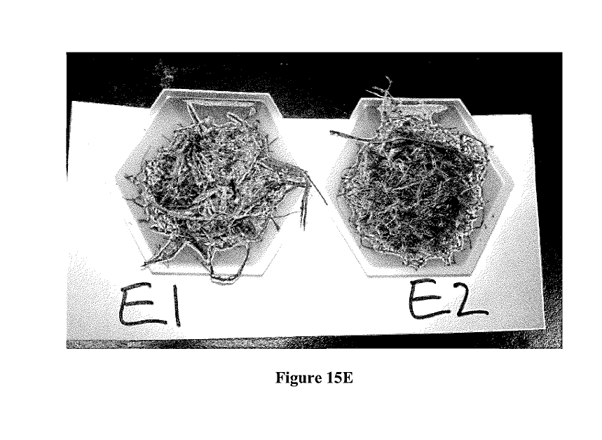

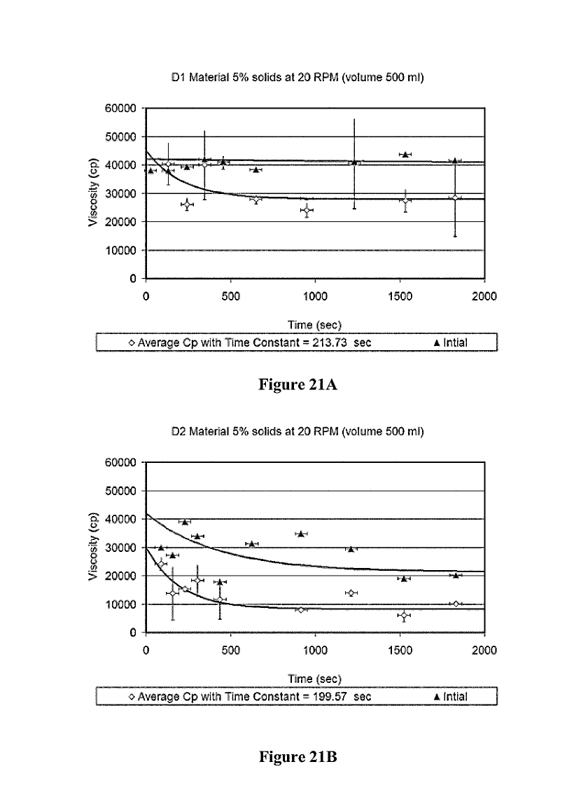

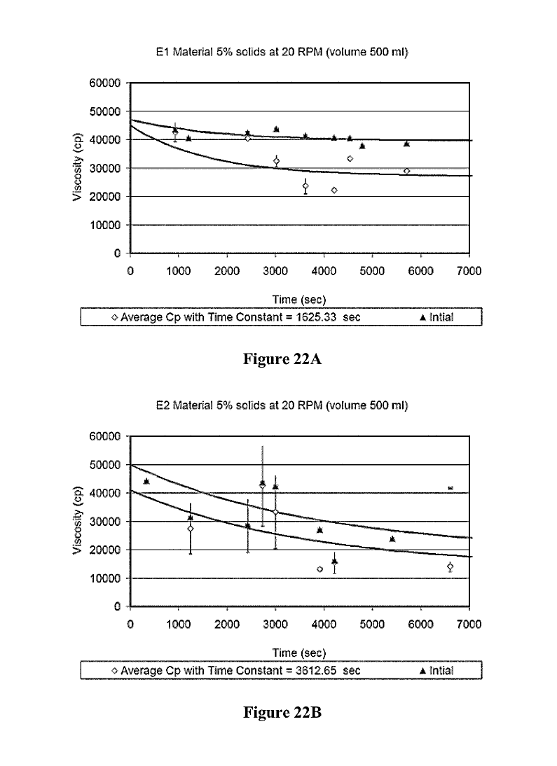

FIGS. 16 to 23 and Table 10 show the decrease in viscosity over time for the different pretreatment conditions. In all cases the steady state viscosity at the start of the reaction is much lower for the steam-exploded samples (2-series) compared to the unexploded samples (1-series). The viscosity decreases after enzyme addition for both steam-exploded and unexploded samples, to varying extents. Most of this reduction occurred within the first 10 minutes of enzyme exposure.

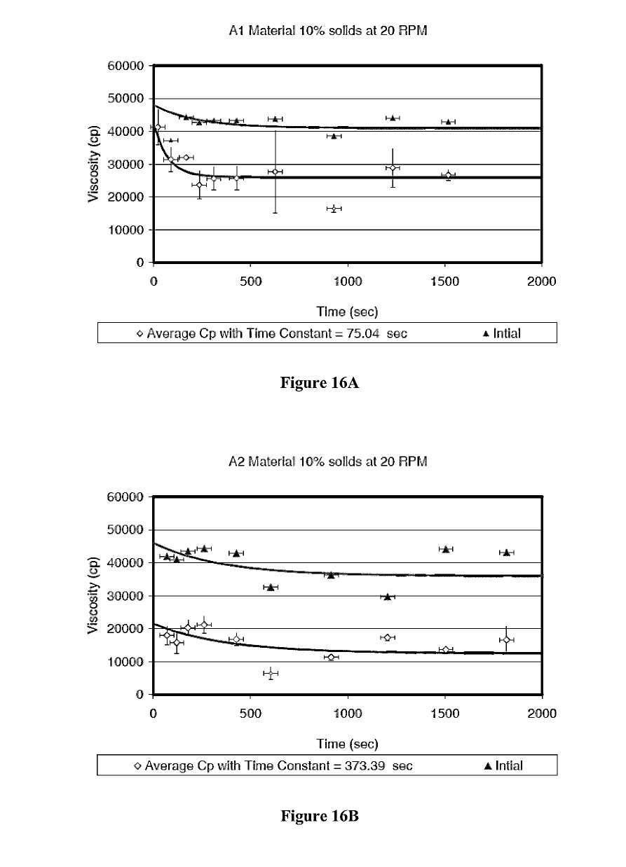

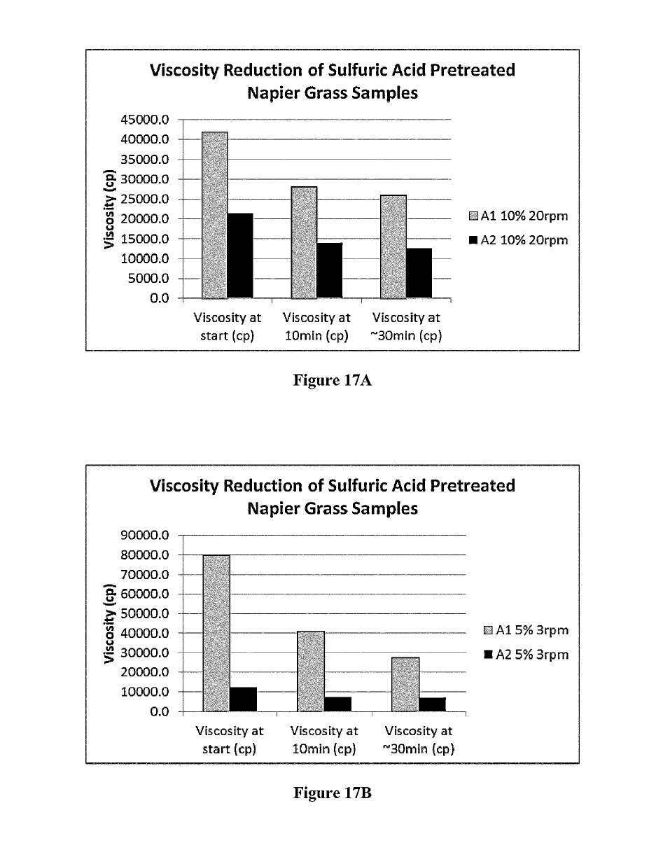

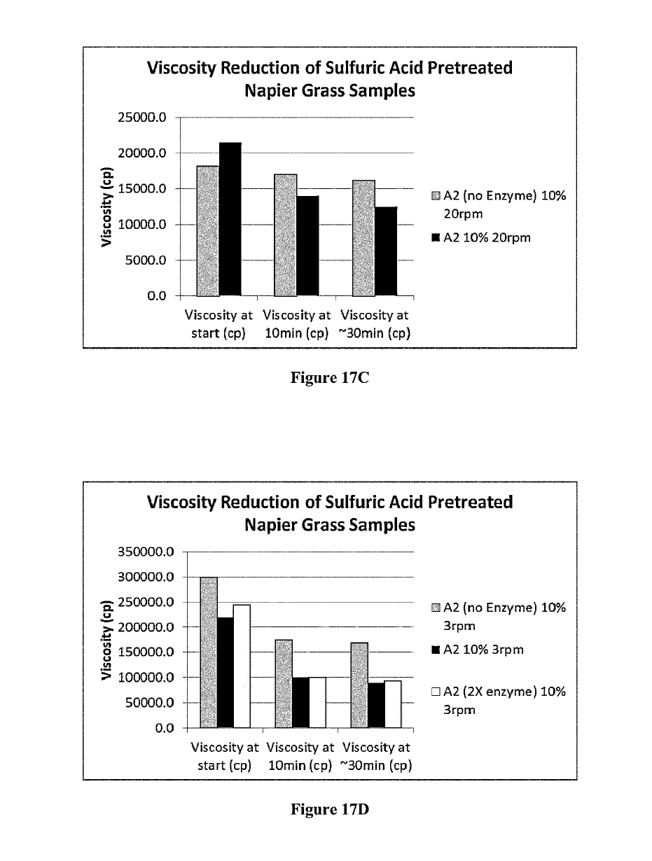

For the sulfuric acid-treated samples tested at 10% solids and 20 rpm rotational speed, viscosity of the unexploded cake was reduced by 38.1% in 30 minutes, reaching a final viscosity of 26000 cp (FIG. 16). For the steam exploded sample tested under the same conditions a reduction of 41.5% was observed, with a final viscosity measurement of 12574 cp, nearly 52% lower than A1 after enzymatic treatment (FIG. 17A). When the same samples were tested at 5% solids, 3 rpm the difference was even more significant. Viscosity of A1 decreased from 80000 cp to 27593 cp over 30 minutes, a 65.5% reduction, while that of A2 decreased from 12500 cp to 7101 cp (FIG. 17B). This is a 43.2% reduction from the starting viscosity, but 74.3% lower than the final viscosity of A1. As shown in FIG. 17C, A2 at 10% solids, 20 rpm showed a 10.8% decrease in viscosity without enzyme over the 30 minute test, compared to the 41.5% reduction with 25 CTU TR1/g solids. The shear-thinning behavior of the material without enzyme is likely due to alignment of the fibers in the reaction vessel as the vane repeatedly passes through the slurry. Addition of twice the enzyme load did not improve the rate or extent of viscosity reduction, as seen in FIG. 17D.

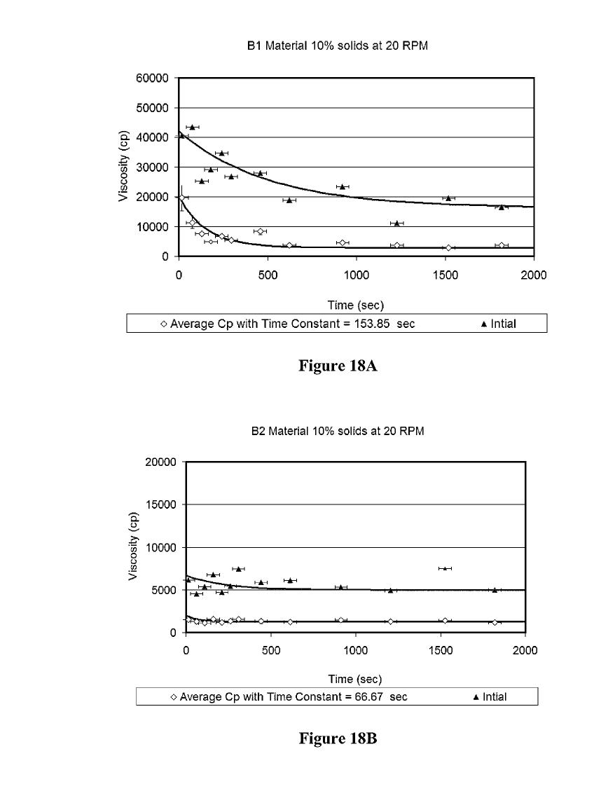

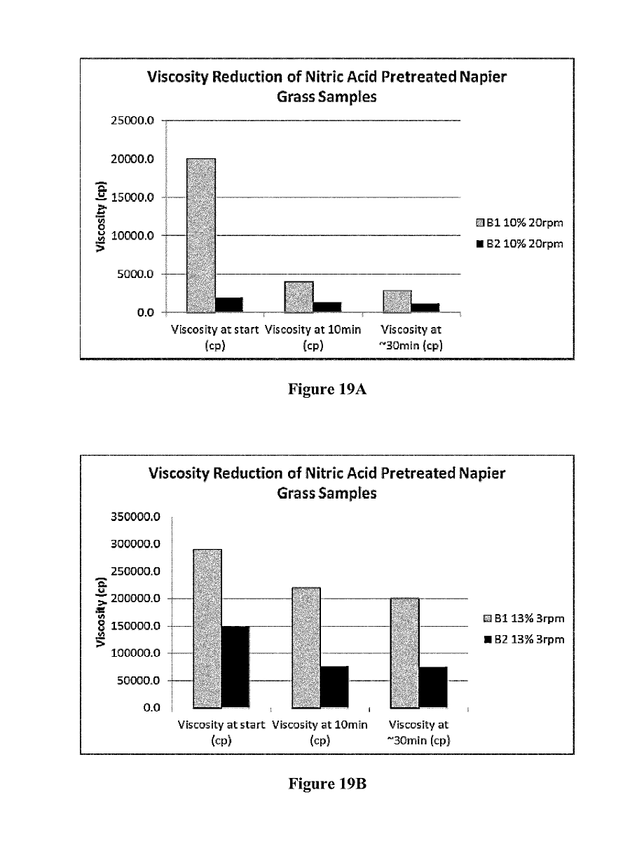

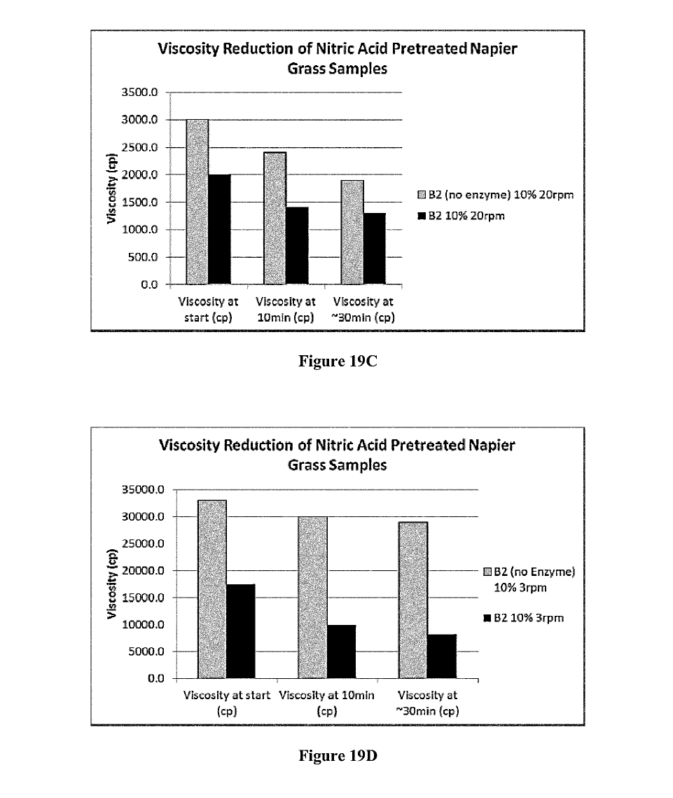

The nitric acid hydrolyzed samples, B1 and B2, had the lowest starting viscosities of all five pretreatment conditions (FIG. 18). At 10% solids, 20 rpm, B1 started at a steady-state viscosity of 20000 cp, only 52.4% the viscosity of the A1 cake under the same test conditions. Addition of enzyme rapidly decreased the viscosity to 4000 cp in the first 10 minutes and to 2900 cp after 30 minutes, an 85.5% decrease (FIG. 19A). Steam-explosion reduced the viscosity dramatically as well. The starting stead-state viscosity of B2 was 90% of the B1 value, at 2000 cP, and was reduced to 1300 cp after 30 minutes of enzyme exposure (FIG. 19A). At higher solids loading (FIG. 19B) viscosity for the unexploded cake was reduced by 31.0%, while viscosity of the steam-exploded cake slurry was reduced by 49.3%. FIGS. 19C and 19D show the comparison of B2 with and without enzyme addition at 10% solids at 20 rpm or 3 rpm rotational speeds. At both speeds the shear-thinning behaviour is observed in the no enzyme sample, while enzyme addition reduces the viscosity even further.

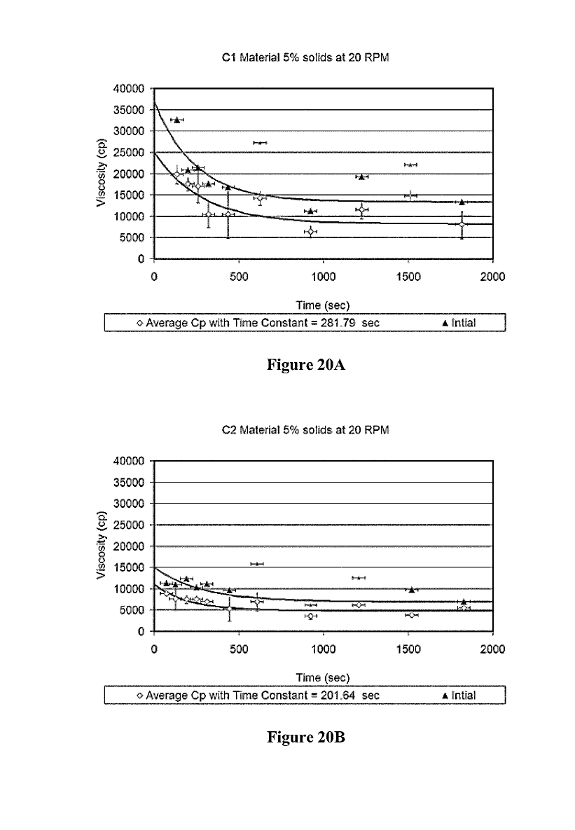

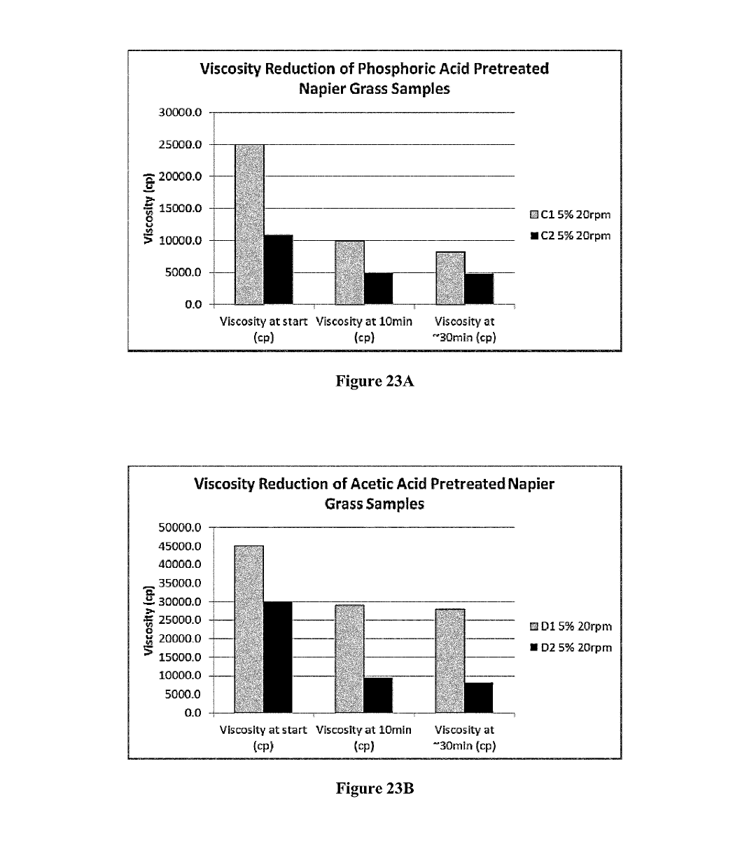

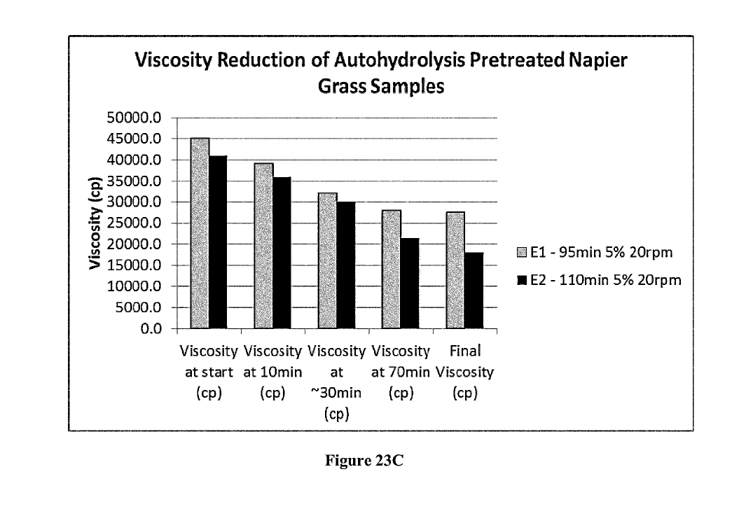

The phosphoric acid (C1/C2), acetic acid (D1/D2), and autohydrolysis pretreatments (E1/E2) resulted in cakes with much higher starting viscosities (FIGS. 20-23). Due to the long fiber lengths and water absorbing capacity observed in these samples, reliable measurements could only be obtained at 5% solids loadings using the Brookfield viscometer. The time courses for phosphoric acid-pretreated, acetic acid-pretreated and autohydrolyzed materials are shown in FIGS. 20, 21 and 22, respectively. For C1, a reduction of 67.1% was observed, with steady-state viscosity decreasing from 25000 cp to 8228 cp over the 30 minute test. C2 started at 11000 cp, already a 56% decrease from the C1 starting measurement, and was reduced to 4821 cP over the same 30 minute reaction time (FIG. 23A). D1 and D2 were even more viscous than C1/C2, but behaved similarly when treated with enzyme. D1 viscosity was reduced by 37.9% from the starting steady-state, while D2 was reduced by 72.7%, to a final viscosity of 8203 cp (FIG. 23B). Finally, the autohydrolysis cakes had the highest starting steady state viscosities at 45000 cp and 41000 cp for E1 and E2, respectively. Viscosity reduction for these samples was slower than the others, and there was less of a difference between the unexploded and steam-exploded samples (FIG. 23C). After 10 minutes of enzyme exposure, viscosity was only reduced by 13.3% and 12.2% for E1 and E2, respectively. After 95 minutes for E1 and 110 minutes for E2, the viscosity was reduced by 38.8% and 56.0% from the starting steady-state values. This corresponded to viscosity measurements of 27545 cp and 18026 cp, higher than any of the acid pretreated samples after 30 minutes of enzyme treatment.

Biomass slurries are non-Newtonian fluids that can exhibit a series of inherent viscous behaviors that change with time, such as shear thinning behavior, shear thickening behavior, and long term particle shape effects. The studies presented herein demonstrate that flowability and mixability are impacted by multiple factors, such as the use of enzymatic liquefaction, pretreatment methods, solids loading, and temperature. The studies further demonstrate that when biomass is pretreated by a combination of enzymatic liquefaction under conditions in which cellulose is not substantially saccharified plus one or more additional pretreatment methods (e.g., steam explosion, acid pretreatment), the combination can act in concert to give additive and in some cases synergistic effects in improving biomass liquefaction. These data suggest a mechanism in which the enzymes and other pretreatment methods operate to modify different bonds in the cellulose that make the biomass more amenable to flow/mixing.

### 7. SPECIFIC EMBODIMENTS AND INCORPORATION BY REFERENCE

Illustrative embodiments of the disclosure are described below in the following numbered paragraphs:
1. A method for producing or processing biomass slurry/pretreating biomass/liquefying biomass/preparing biomass for downstream processing, comprising mixing biomass which has been subject to steam explosion with an aqueous liquid in the presence of one or more hydrolyzing proteins under conditions that:
   (a) are unfavorable for enzymatic saccharification by said one or more hydrolyzing proteins; and/or
   (b) result in less than 40%, less than 30%, less than 20% or less than 10% glucan (e.g., glucose and/or cellobiose) saccharification by said one or more hydrolyzing proteins; and/or
   (c) require at least 10%, at least 20%, at least 30%, or at least 40% less power to mix the biomass with the aqueous liquid as compared to mixing biomass and aqueous liquid in the absence of said hydrolyzing proteins over a 2-, 5-, 10-, 15- or 20-minute period; and/or
   (d) permit mixing of a slurry containing at least 10%, at least 20%, at least 30%, or at least 40% more biomass solids without increasing power usage as compared to a slurry mixed in the absence of said hydrolyzing proteins,
   thereby producing or processing biomass slurry/pretreating biomass/liquefying biomass/preparing biomass for downstream processing.
2. The method of embodiment 1, wherein the conditions yield 10% or less, 8% or less, 6% or less, 5% or less, 4% or less, 3% or less, or 2% or less of the theoretical yield of (i) glucose, (ii) xylose, (iii) cellobiose, (iv) both glucose and xylose, (v) both glucose and cellobiose, (vi) both xylose and cellobiose, or (vii) each of glucose, xylose and cellobiose in the biomass.
3. The method of embodiment 1 or embodiment 2, wherein the conditions are effective to reduce the viscosity of the biomass by at least 10%, by at least 20%, by at least 30%, by at least 40% or by at least 50%.
4. The method of any one of embodiments 1 to 3, wherein the steam explosion has been carried out under conditions that reduce the viscosity of the biomass by at least 10%, by at least 20%, by at least 30%, by at least 40% or by at least 50%.
5. The method of any one of embodiments 1 to 4, wherein the one or more hydrolyzing proteins are at a dose of 5 μg to 40 mg, 5 μg to 30 mg or 5 μg to 20 mg protein or 10-250 CTUs per gram dry weight of biomass.
6. The method of any one of embodiments 1 to 5, wherein the biomass has been subject to acid pretreatment.
7. The method of any one of embodiments 1 to 6, wherein the mixing is carried out at a temperature of 50° C. to 100° C., 60° C. to 100° C., or 50° C. to 100° C., 60° C. to 100° C., or 50° C. to 80° C.
8. The method of embodiment 7, wherein the mixing is carried out at a temperature of 65° C. to 75° C., 62° C. to 72° C., or 62° C. to 75° C.
9. The method of any one of embodiments 1 to 8, wherein the mixing is carried out for a period of at least 0.25 minutes, at least 0.5 minute, at least 1 minute or at least 2 minutes, at least 5 minutes, at least 10 minutes, or at least 15 minutes.
10. The method of any one of embodiments 1 to 9, wherein the mixing is carried out for a period of up to 30 minutes, up to 1 hour or up to 1.5 hours.
11. The method of any one of embodiments 1 to 10, wherein the biomass and the aqueous liquid are at a 1:1 to 1:7, 1:2 to 1:6, 1:1 to 1:7, 1:2 to 1:6, 1:2.5 to 1:5.7, 1:3.33 to 1:5.7, or 1:4 to 1:5.7 solid:liquid weight ratio.
12. A method for continuous production or processing of biomass slurry/pretreating biomass/liquefying biomass/preparing biomass for downstream processing, comprising:
   (a) combining biomass which has been subject to steam explosion, an aqueous liquid and one or more hydrolyzing proteins into a vessel comprising a biomass slurry, wherein the biomass, an aqueous liquid and one or more hydrolyzing proteins are introduced into the vessel at a rate in which the slurry viscosity in the vessel is maintained,
   (b) simultaneously pumping slurry out of the vessel at a rate that maintains the slurry volume in the vessel;
   wherein the conditions in the vessel:
      (i) are unfavorable for enzymatic saccharification by said one or more hydrolyzing proteins; and/or
      (ii) result in less than 40%, less than 30%, less than 20% or less than 10% glucan (e.g., glucose and/or cellobiose) saccharification by said one or more hydrolyzing proteins; and/or
      (iii) require at least 10%, at least 20%, at least 30%, or at least 40% less power to mix the biomass with the aqueous liquid as compared to mixing biomass and aqueous liquid in the absence of said hydrolyzing proteins over the residence time of the biomass in the vessel; and/or
      (iv) permit mixing of a slurry containing at least 10%, at least 20%, at least 30%, or at least 40% more biomass solids without increasing power usage as compared to a slurry mixed in the absence of said hydrolyzing proteins,
   thereby continuously producing or processing biomass slurry/pretreating biomass/liquefying biomass/preparing biomass for downstream processing.
13. The method of embodiment 12, wherein the steam explosion has been carried out under conditions that reduce the viscosity of the biomass by at least 10%, by at least 20%, by at least 30%, by at least 40% or by at least 50%.
14. The method of embodiment 12 or embodiment 13, wherein the one or more hydrolyzing proteins are at a dose of 5 μg to 40 mg, 5 μg to 30 mg or 5 μg to 20 mg protein or 10-250 CTUs per gram dry weight of biomass.
15. The method of any one of embodiments 12 to 14, wherein the biomass has been subject to acid pretreatment.
16. The method of any one of embodiments 12 to 15, wherein the vessel is maintained at a temperature of 50° C. to 100° C., 60° C. to 100° C., or 50° C. to 80° C.
17. The method of embodiment 16, wherein the vessel is maintained at a temperature of 65° C. to 75° C., 62° C. to 72° C., or 62° C. to 75° C.
18. The method of any one of embodiments 12 to 17, wherein the biomass slurry comprises at least 5%, at least 8% or at least 10% weight solids pretreated with one or more hydrolyzing proteins in a dose of 5 μg to 40 mg, 5 μg to 30 mg or 5 μg to 20 mg protein or 10-250 CTUs per gram dry weight of biomass.
19. The method of embodiment 18, wherein the solids are from biomass which has been subject to steam explosion prior to said pretreatment with hydrolyzing proteins.
20. The method of embodiment 19, wherein the steam explosion prior to pretreatment has been carried out under conditions that reduce the viscosity of the biomass by at least 10%, by at least 20%, by at least 30%, by at least 40% or by at least 50%.
21. The method of any one of embodiments 12 to 20, wherein the vessel is a continuous stirred tank reactor (“CSTR”).
22. The method of any one of embodiments 12 to 20, wherein the vessel is a plug flow reactor (“PFR”).
23. The method of any one of embodiments 12 to 22, which comprises continuously producing or processing biomass slurry/pretreating biomass/liquefying biomass/preparing biomass for downstream processing for a period of time of at least 12 hours or at least 18 hours.
24. The method of embodiment 23, which comprises continuously producing or processing biomass slurry/pretreating biomass/liquefying biomass/preparing biomass for downstream processing for a period of time of up to 24 hours, up to 36 hours, up to 48 hours, up to 72 hours, up to 96 hours, up to 1 week, up to 2 weeks, up to 3 weeks, up to 1 month, up to 6 months, or up to 1 year.
25. The method of embodiment 23 or embodiment 24, in which the vessel is not cleaned during said period of time.
26. The method of any one of embodiments 12 to 25, in which 3% to 10% of the slurry volume is pumped out of the vessel every minute.
27. The method of any one of embodiments 12 to 26, in which the slurry has a residence time of less than 2 hours in the vessel.
28. The method of embodiment 27, wherein the slurry has a residence time of 2 minutes to 30 minutes in the vessel.
29. The method of any one of embodiments 12 to 28, further comprising, prior to step (a), forming said biomass slurry.
30. The method of embodiment 29, wherein forming said biomass slurry comprises combining in said vessel biomass which has been subject to steam explosion with an aqueous liquid in the presence of one or more hydrolyzing proteins.
31. The method of embodiment 30, wherein the steam explosion has been carried out under conditions that reduce the viscosity of the biomass by at least 10%, by at least 20%, by at least 30%, by at least 40% or by at least 50%.
32. The method of embodiment 30 or embodiment 31, wherein said one or more hydrolyzing proteins are at a dose of 5 μg to 40 mg, 5 μg to 30 mg or 5 μg to 20 mg protein or 10-250 CTUs per gram dry weight of biomass.
33. The method of any one of embodiments 30 to 32, wherein the biomass and the aqueous liquid are combined at a 1:1 to 1:7, 1:2 to 1:6, 1:2.5 to 1:5.7, 1:3.33 to 1:5.7, or 1:4 to 1:5.7 solid:liquid weight ratio.
34. The method of any one of embodiments 30 to 33, wherein the vessel is maintained at a temperature of 50° C. to 100° C., 60° C. to 100° C., or 50° C. to 80° C.
35. The method of embodiment 35, wherein the vessel is maintained at a temperature in the range of 65° C. to 75° C., 62° C. to 72° C., or 62° C. to 75° C.
36. The method of any one of embodiments 30 to 35, which further comprises agitating the vessel contents during slurry formation.
37. The method of embodiment 36, wherein the vessel contents are agitated for a period of at least 0.5 minute, at least 1 minute or at least 2 minutes, at least 5 minutes, at least 10 minutes, or at least 15 minutes.
38. The method of embodiment 36 or embodiment 37, wherein the vessel contents are agitated for a period of up to 1 hour or up to 1.5 hours.
39. The method of any one of embodiments 30 to 38, wherein the biomass has been subject to acid pretreatment.
40. A method for producing or processing biomass slurry/pretreating biomass/liquefying biomass/preparing biomass for downstream processing, comprising mixing biomass with an aqueous liquid in the presence of one or more hydrolyzing proteins in a dose of 5 μg to 40 mg, 5 μg to 30 mg or 5 μg to 20 mg protein or 10-250 CTUs per gram dry weight of biomass at 50° C. to 100° C., 60° C. to 100° C., 50° C. to 80° C., 65° C. to 75° C., 62° C. to 72° C., or 62° C. to 75° C. for a period of at least 0.25 minutes, at least 0.5 minute, at least 1 minute or at least 2 minutes, at least 5 minutes, at least 10 minutes, or at least 15 minutes, thereby producing or processing biomass slurry/pretreating biomass/liquefying biomass/preparing biomass for downstream processing.
41. The method of embodiment 40, wherein the biomass has been subject to steam explosion.
42. The method of embodiment 41, wherein the steam explosion has been carried out under conditions that reduce the viscosity of the biomass by at least 10%, by at least 20%, by at least 30%, by at least 40% or by at least 50%.
43. The method of any one of embodiments 40 to 42, wherein the mixing is carried out for a period of up to 30 minutes, up to 1 hour or up to 1.5 hours.
44. The method of any one of embodiments 40 to 43, wherein the biomass and the aqueous liquid are at a 1:1 to 1:7, 1:2 to 1:6, 1:2.5 to 1:5.7, 1:3.33 to 1:5.7, or 1:4 to 1:5.7 solid:liquid weight ratio.
45. The method of any one of embodiments 40 to 44, wherein the biomass has been subject to acid pretreatment.
46. A method for producing or processing biomass slurry/pretreating biomass/liquefying biomass/preparing biomass for downstream processing, comprising treating a biomass slurry with one or more hydrolyzing proteins in a dose of 5 μg to 40 mg, 5 μg to 30 mg or 5 μg to 20 mg protein or 10-250 CTUs per gram dry weight of biomass at 65° C. to 75° C., 62° C. to 72° C., or 62° C. to 75° C. for a period of at least 0.25 minutes, at least 0.5 minute, at least 1 minute or at least 2 minutes, at least 5 minutes, at least 10 minutes, or at least 15 minutes, thereby biomass slurry/pretreating biomass/liquefying biomass/preparing biomass for downstream processing.
47. The method of embodiment 46, wherein the biomass has been subject to steam explosion.
48. The method of embodiment 47, wherein the steam explosion has been carried out under conditions that reduce the viscosity of the biomass by at least 10%, by at least 20%, by at least 30%, by at least 40% or by at least 50%.
49. The method of any one of embodiments 46 to 48, wherein the treatment is carried out for a period of up to 1 hour or up to 1.5 hours.
50. The method of any one of embodiments 46 to 49, wherein the slurry comprises 15%-40%, 15%-30% or 15%-25% by weight solids.
51. The method of any one of embodiments 46 to 50, wherein the biomass has been subject to acid pretreatment.
52. A method for producing or processing biomass slurry/pretreating biomass/liquefying biomass/preparing biomass for downstream processing, comprising mixing biomass with an aqueous liquid at a 1:1 to 1:7, 1:2 to 1:6, 1:2.5 to 1:5.7, 1:3.33 to 1:5.7, or 1:4 to 1:5.7 solid:liquid weight ratio in the presence of one or more hydrolyzing proteins in a dose of 5 μg to 40 mg, 5 μg to 30 mg or 5 μg to 20 mg protein or 10-250 CTUs per gram dry weight of biomass at 50° C. to 100° C., 60° C. to 100° C., or 50° C. to 80° C. for a period of at least 0.25 minutes, at least 0.5 minute, at least 1 minute or at least 2 minutes, at least 5 minutes, at least 10 minutes, or at least 15 minutes, thereby producing or processing biomass slurry/pretreating biomass/liquefying biomass/preparing biomass for downstream processing.
53. The method of embodiment 52, wherein the biomass has been subject to steam explosion.
54. The method of embodiment 53, wherein the steam explosion has been carried out under conditions that reduce the viscosity of the biomass by at least 10%, by at least 20%, by at least 30%, by at least 40% or by at least 50%.
55. The method of any one of embodiments 52 to 54, wherein the mixing is carried out for a period of up to 1 hour or up to 1.5 hours.
56. The method of any one of embodiments 52 to 55, which is performed at a temperature in the range of 65° C. to 75° C., 62° C. to 72° C., or 62° C. to 75° C.
57. The method of any one of embodiments 52 to 56, wherein the biomass has been subject to acid pretreatment.
58. A method for producing or processing biomass slurry/pretreating biomass/liquefying biomass/preparing biomass for downstream processing, comprising treating a biomass slurry comprising 15%-40%, 15%-30% or 15%-25% by weight solids with one or more hydrolyzing proteins in a dose of 5 μg to 40 mg, 5 μg to 30 mg or 5 μg to 20 mg protein or 10-250 CTUs per gram dry weight of biomass at 50° C. to 100° C., 60° C. to 100° C., or 50° C. to 80° C. for a period of at least 0.25 minutes, at least 0.5 minute, at least 1 minute or at least 2 minutes, at least 5 minutes, at least 10 minutes, or at least 15 minutes, thereby producing or processing biomass slurry/pretreating biomass/liquefying biomass/preparing biomass for downstream processing.
59. The method of embodiment 58, wherein the biomass has been subject to steam explosion.
60. The method of embodiment 59, wherein the steam explosion has been carried out under conditions that reduce the viscosity of the biomass by at least 10%, by at least 20%, by at least 30%, by at least 40% or by at least 50%.
61. The method of any one of embodiments 58 to 60, wherein the treatment is carried out for a period of up to 1 hour or up to 1.5 hours.
62. The method of any one of embodiments 58 to 62, which is performed at a temperature in the range of 65° C. to 75° C., 62° C. to 72° C., or 62° C. to 75° C.
63. The method of any one of embodiments 58 to 62, wherein the biomass has been subject to acid pretreatment.
64. A method for producing or processing biomass slurry/pretreating biomass/liquefying biomass/preparing biomass for downstream processing, comprising mixing biomass with an aqueous liquid in the presence of one or more hydrolyzing proteins in a dose of 5 μg to 40 mg, 5 μg to 30 mg or 5 μg to 20 mg protein or 10-250 CTUs per gram dry weight of biomass at 50° C. to 100° C., 60° C. to 100° C., or 50° C. to 80° C. for a period of at least 0.25 minutes, at least 0.5 minute, at least 1 minute or at least 2 minutes, at least 5 minutes, at least 10 minutes, or at least 15 minutes and up to one hour or up to 1.5 hours, thereby producing or processing biomass slurry/pretreating biomass/liquefying biomass/preparing biomass for downstream processing.
65. The method of embodiment 64, wherein the biomass has been subject to steam explosion.
66. The method of embodiment 65, wherein the steam explosion has been carried out under conditions that reduce the viscosity of the biomass by at least 10%, by at least 20%, by at least 30%, by at least 40% or by at least 50%.
67. The method of any one of embodiments 64 to 66, wherein the biomass and the aqueous liquid are at a 1:1 to 1:7, 1:2 to 1:6, 1:2.5 to 1:5.7, 1:3.33 to 1:5.7, or 1:4 to 1:5.7 solid:liquid weight ratio.
68. The method of any one of embodiments 64 to 67, which is performed at a temperature in the range of 65° C. to 75° C., 62° C. to 72° C., or 62° C. to 75° C.
69. The method of any one of embodiments 64 to 68, wherein the biomass has been subject to acid pretreatment.
70. A method for producing or processing biomass slurry/pretreating biomass/liquefying biomass/preparing biomass for downstream processing, comprising treating a biomass slurry with one or more hydrolyzing proteins in a dose of 5 μg to 40 mg, 5 μg to 30 mg or 5 μg to 20 mg protein or 10-250 CTUs per gram dry weight of biomass at 50° C. to 100° C., 60° C. to 100° C., or 50° C. to 80° C. for a period of at least 0.25 minutes, at least 0.5 minute, at least 1 minute or at least 2 minutes, at least 5 minutes, at least 10 minutes, or at least 15 minutes and up to one hour, thereby producing or processing biomass slurry/pretreating biomass/liquefying biomass/preparing biomass for downstream processing.
71. The method of embodiment 70, wherein the biomass has been subject to steam explosion.
72. The method of embodiment 71, wherein the steam explosion has been carried out under conditions that reduce the viscosity of the biomass by at least 10%, by at least 20%, by at least 30%, by at least 40% or by at least 50%.
73. The method of any one of embodiments 70 to 72, wherein the slurry comprises 15%-40%, 15%-30% or 15%-25% by weight solids.
74. The method of any one of embodiments 70 to 73, which is performed at a temperature in the range of 65° C. to 75° C., 62° C. to 72° C., or 62° C. to 75° C.
75. The method of any one of embodiments 70 to 74, wherein the biomass has been subject to acid pretreatment.
76. A method for producing or processing biomass slurry/pretreating biomass/liquefying biomass/preparing biomass for downstream processing, comprising mixing acid-pretreated biomass with an aqueous liquid in the presence of one or more hydrolyzing proteins in a dose of 5 μg to 40 mg, 5 μg to 30 mg or 5 μg to 20 mg protein or 10-250 CTUs per gram dry weight of biomass at 50° C. to 100° C., 60° C. to 100° C., or 50° C. to 80° C. for a period of at least 0.25 minutes, at least 0.5 minute, at least 1 minute or at least 2 minutes, at least 5 minutes, at least 10 minutes, or at least 15 minutes, thereby producing or processing biomass slurry/pretreating biomass/liquefying biomass/preparing biomass for downstream processing.
77. The method of embodiment 76, wherein the biomass has been subject to steam explosion.
78. The method of embodiment 77, wherein the steam explosion has been carried out under conditions that reduce the viscosity of the biomass by at least 10%, by at least 20%, by at least 30%, by at least 40% or by at least 50%.
79. The method of any one of embodiments 76 to 78, wherein the biomass and the aqueous liquid are at a 1:1 to 1:7, 1:2 to 1:6, 1:2.5 to 1:5.7, 1:3.33 to 1:5.7, or 1:4 to 1:5.7 solid:liquid weight ratio.
80. The method of any one of embodiments 76 to 79, which is performed at a temperature in the range of 65° C. to 75° C., 62° C. to 72° C., or 62° C. to 75° C.
81. The method of any one of embodiments 76 to 80, wherein the mixing is carried out for a period of up to 1 hour or up to 1.5 hours.
82. A method for producing or processing biomass slurry/pretreating biomass/liquefying biomass/preparing biomass for downstream processing, comprising treating a acid-pretreated biomass slurry comprising 15%-40%, 15%-30% or 15%-25% weight solids with one or more hydrolyzing proteins in a dose of 5 μg to 40 mg, 5 μg to 30 mg or 5 μg to 20 mg protein or 10-250 CTUs per gram dry weight of biomass at 50° C. to 100° C., 60° C. to 100° C., or 50° C. to 80° C. for a period of at least 0.25 minutes, at least 0.5 minute, at least 1 minute or at least 2 minutes, at least 5 minutes, at least 10 minutes, or at least 15 minutes, thereby producing or processing biomass slurry/pretreating biomass/liquefying biomass/preparing biomass for downstream processing.
83. The method of embodiment 82, wherein the biomass has been subject to steam explosion.
84. The method of embodiment 83, wherein the steam explosion has been carried out under conditions that reduce the viscosity of the biomass by at least 10%, by at least 20%, by at least 30%, by at least 40% or by at least 50%.
85. The method of any one of embodiments 82 to 84, wherein the slurry comprises 15%-40%, 15%-30% or 15%-25% by weight solids.
86. The method of any one of embodiments 82 to 85, which is performed at a temperature in the range of 65° C. to 75° C., 62° C. to 72° C., or 62° C. to 75° C.
87. The method of any one of embodiments 82 to 86, wherein the mixing is carried out for a period of up to 1 hour or up to 1.5 hours.
88. A method for continuous production or processing of biomass slurry/pretreating biomass/liquefying biomass/preparing biomass for downstream processing, comprising:
   (a) combining biomass, an aqueous liquid and one or more hydrolyzing proteins into a vessel maintained at 65° C. to 75° C., 62° C. to 72° C., or 62° C. to 75° C., said vessel comprising a biomass slurry comprising 15%-40%, 15%-30% or 15%-25% weight solids pretreated with one or more hydrolyzing proteins in a dose of 5 μg to 40 mg, 5 μg to 30 mg or 5 μg to 20 mg protein or 10-250 CTUs per gram dry weight of biomass, wherein the biomass, an aqueous liquid and one or more hydrolyzing proteins are introduced into the vessel at a rate in which the slurry viscosity in the vessel is maintained,
   (b) simultaneously pumping slurry out of the vessel at a rate that maintains the slurry volume in the vessel;
   thereby continuously producing or processing biomass slurry/pretreating biomass/liquefying biomass/preparing biomass for downstream processing.
89. The method of embodiment 88, wherein the biomass has been subject to steam explosion.
90. The method of embodiment 89, wherein the steam explosion has been carried out under conditions that reduce the viscosity of the biomass by at least 10%, by at least 20%, by at least 30%, by at least 40% or by at least 50%.
91. The method of any one of embodiments 88 to 90, wherein the vessel is a continuous stirred tank reactor (“CSTR”).
92. The method of any one of embodiments 88 to 90, wherein the vessel is a plug flow reactor (“PFR”).
93. The method of any one of embodiments 88 to 92, which comprises continuously producing or processing biomass slurry/pretreating biomass/liquefying biomass/preparing biomass for downstream processing for a period of time of at least 12 hours or at least 18 hours.
94. The method of embodiment 93, which comprises continuously producing or processing biomass slurry/pretreating biomass/liquefying biomass/preparing biomass for downstream processing for a period of time of up to 24 hours, up to 36 hours, up to 48 hours, up to 72 hours, up to 96 hours, up to 1 week, up to 2 weeks, up to 3 weeks, up to 1 month, up to 6 months, or up to 1 year.
95. The method of embodiment 93 or embodiment 94, in which the vessel is not cleaned during said period of time.
96. The method of any one of embodiments 88 to 95, in which 3% to 10% of the slurry volume is pumped out of the vessel every minute.
97. The method of any one of embodiments 88 to 96, in which the slurry has a residence time of less than 2 hours in the vessel.
98. The method of embodiment 97, wherein the slurry has a residence time of 2 minutes to 30 minutes in the vessel.
99. The method of any one of embodiments 88 to 98, further comprising, prior to step (a), forming said biomass slurry.
100. The method of embodiment 99, wherein forming said biomass slurry comprises combining in said vessel biomass with an aqueous liquid in the presence of one or more hydrolyzing proteins,
101. The method of embodiment 100, wherein the biomass has been subject to steam explosion.
102. The method of embodiment 101, wherein the steam explosion has been carried out under conditions that reduce the viscosity of the biomass by at least 10%, by at least 20%, by at least 30%, by at least 40% or by at least 50%.
103. The method of any one of embodiments 100 to 102, wherein said one or more hydrolyzing proteins are at a dose of 5 μg to 40 mg, 5 μg to 30 mg or 5 μg to 20 mg protein or 10-250 CTUs per gram dry weight of biomass.
104. The method of any one of embodiments 100 to 103, wherein the biomass and the aqueous liquid are combined at a 1:1 to 1:7, 1:2 to 1:6, 1:2.5 to 1:5.7, 1:3.33 to 1:5.7, or 1:4 to 1:5.7 solid:liquid weight ratio.
105. The method of any one of embodiments 100 to 104, wherein the vessel is at a temperature of 50° C. to 100° C., 60° C. to 100° C., or 50° C. to 80° C.
106. The method of embodiment 105, wherein the vessel is at a temperature in the range of 65° C. to 75° C., 62° C. to 72° C., or 62° C. to 75° C.
107. The method of any one of embodiments 99 to 106, which further comprises agitating the vessel contents during slurry formation.
108. The method of embodiment 107, wherein the vessel contents are agitated for a period of at least 0.5 minute, at least 1 minute or at least 2 minutes, at least 5 minutes, at least 10 minutes, or at least 15 minutes.
109. The method of embodiment 106 or embodiment 107, wherein the vessel contents are agitated for a period of up to 1 hour or up to 1.5 hours.
110. The method of any one of embodiments 100 to 109, wherein the biomass has been subject to acid pretreatment.
111. A method of producing or processing biomass slurry/pretreating biomass/liquefying biomass/preparing biomass for downstream processing, comprising combining in a vessel biomass with an aqueous liquid in the presence of one or more hydrolyzing proteins.
112. The method of embodiment 111, wherein the biomass has been subject to steam explosion.
113. The method of embodiment 112, wherein the steam explosion has been carried out under conditions that reduce the viscosity of the biomass by at least 10%, by at least 20%, by at least 30%, by at least 40% or by at least 50%.
114. The method of any one of embodiments to 111 to 113, wherein said one or more hydrolyzing proteins are at a dose of 5 ng to 40 mg, 5 ng to 30 mg or 5 ng to 20 mg protein or 10-250 CTUs per gram dry weight of biomass.
115. The method of any one of embodiments 111 to 114, wherein the biomass and the aqueous liquid are combined at a 1:1 to 1:7, 1:2 to 1:6, 1:2.5 to 1:5.7, 1:3.33 to 1:5.7, or 1:4 to 1:5.7 solid:liquid weight ratio.
116. The method of any one of embodiments 111 to 115, wherein the vessel is at a temperature of 50° C. to 100° C., 60° C. to 100° C., or 50° C. to 80° C.
117. The method of embodiment 116, wherein the slurry in the vessel is formed at a temperature in the range of 65° C. to 75° C., 62° C. to 72° C., or 62° C. to 75° C.
118. The method of any one of embodiments 111 to 118, which further comprises agitating the vessel contents during slurry formation.
119. The method of embodiment 118, wherein the vessel contents are agitated for a period of at least 0.5 minute, at least 1 minute or at least 2 minutes, at least 5 minutes, at least 10 minutes, or at least 15 minutes.
120. The method of embodiment 118 or embodiment 119, wherein the mixing is carried out the vessel contents are agitated for a period of up to 1 hour or up to 1.5 hours.
121. The method of any one of embodiments 111 to 120, wherein the biomass has been subject to acid pretreatment.
122. The method of any one of embodiments 111 to 121, wherein the vessel is a CSTR or a PFR.
123. The method of any one of embodiments 1 to 122, which is carried out in a vessel whose temperature is maintained by a plate and frame heat exchanger and/or a spiral heat exchanger.
124. The method of any one of embodiments 1 to 123, wherein the one or more hydrolyzing proteins comprise one or more cellulases.
125. The method of embodiment 124, wherein the one or more cellulases comprise one or more T. reesei cellobiohydrolases, endoglucanases and/or β-glucosidases.
126. The method of any one of embodiments 1 to 125, wherein the one or more hydrolzying proteins are in a dose of 25-250 CTUs per gram dry weight of biomass.
127. The method of any one of embodiments 1 to 126, wherein producing or processing biomass slurry/pretreating biomass/liquefying biomass/preparing biomass for downstream processing is carried under conditions that result in saccharification of less than 4% of the cellulose and hemicellulose in the biomass by said by said one or more hydrolyzing proteins.
128. The method of embodiment 127, wherein producing or processing biomass slurry/pretreating biomass/liquefying biomass/preparing biomass for downstream processing is carried under conditions that result in saccharification of less than 3% of the cellulose and hemicellulose in the biomass by said one or more hydrolyzing proteins.
129. The method of method of any one of embodiments 12 to 126, which is carried out under conditions that yield 10% or less, 8% or less, 6% or less, 5% or less, 4% or less, 3% or less, or 2% or less of the theoretical yield of (i) glucose, (ii) xylose, (iii) cellobiose, (iv) both glucose and xylose, (v) both glucose and cellobiose, (vi) both xylose and cellobiose, or (vii) each of glucose, xylose and cellobiose in the biomass.
130. The method of any one of embodiments 1 to 129, wherein the biomass is lignocellulosic biomass.
131. The method of embodiment 130, wherein the biomass comprises one or more of Napier grass, energy cane, sorghum, giant reed, sugar beet, switchgrass, bagasse, rice straw, miscanthus, switchgrass, wheat straw, wood, wood waste, paper, paper waste, agricultural waste, municipal waste, birchwood, oat spelt, corn stover, eucalyptus, willow, hybrid poplar, short-rotation woody crop, conifer softwood, crop residue.
132. The method of any one of embodiments 1 to 131, wherein the aqueous liquid is water.
133. The method of any one of embodiments 1 to 131, wherein the aqueous liquid comprises hemicelluloses.
134. The method of any one of embodiments 46 to 51, 58 to 63, 70 to 75, and 82 to 122, wherein the slurry comprises hemicelluloses.
135. The method of any one of embodiments 1 to 134, wherein the one or more hydrolyzing proteins are one or more cellulases or a cellulase cocktail.
136. The method of any one of embodiments 1 to 135, which is carried out with agitation of the biomass slurry.
137. The method of embodiment 136, wherein the biomass slurry is agitated with a paddle mixer, magnetic stirrer, shaker, pump, or homogenizer.
138. The method of any one of embodiments 1 to 137, which is carried out under conditions that result in a biomass slurry that requires at least 5%, at least 10%, at least 20%, at least 30%, at least 40%, or at least 50% less power to agitate as compared to a biomass slurry not treated with said one or more hydrolyzing proteins.
139. The method of any one of embodiments 1 to 138, further comprising subjecting the biomass to pretreatment.
140. The method of any one of embodiments 1 to 139, further comprising processing the pretreated biomass in a screw press.
141. The method of any one of embodiments 6, 15, 39, 45, 51, 57, 63, 69, 75, 110, and 121, wherein the acid is sulfuric acid.
142. The method of any one of embodiments 6, 15, 39, 45, 51, 57, 63, 69, 75, 110, and 121, wherein the acid is nitric acid.
143. The method of any one of embodiments 6, 15, 39, 45, 51, 57, 63, 69, 75, 110, and 121, wherein the acid is acetic acid.
144. The method of any one of embodiments 6, 15, 39, 45, 51, 57, 63, 69, 75, 110, and 121, wherein the acid is phosphoric acid.
145. The method of any one of embodiments 6, 15, 39, 45, 51, 57, 63, 69, 75, 110, 121 and 141 to 144, further comprising carrying out said acid pretreatment step.
146. The method of embodiment 145, further comprising subjecting the biomass to steam explosion.
147. The method of embodiment 146, wherein the steam explosion is carried out under a pressure of 50-400 psig, 50-300 psig, 50-250 psig, 75-200 psig or 75-150 psi, a temperature of 150-250° C. or 190-210° C. and for a time period ranging from 0.1-10 minutes, 0.25-8 minutes, from 0.5-2 minutes or from 1-5 minutes.
148. The method of embodiment 146 or 147, wherein the steam explosion precedes the acid pretreatment step.
149. The method of embodiment 146 or 147, wherein the acid pretreatment step precedes the steam explosion.
150. The method of any one of embodiments 1 to 39, 41, 42, 47, 48, 53, 54, 59, 60, 65, 66, 71, 72, 77, 78, 83, 84, 89, 90, 101, 102, 112 and 113, further comprising subjecting the biomass to steam explosion.
151. The method of embodiment 150, wherein the steam explosion is carried out under a pressure of 50-400 psig, 50-300 psig, 50-250 psig, 75-200 psig or 75-150 psi, a temperature of 150-250° C. or 190-210° C. and for a time period ranging from 0.1-10 minutes, 0.25-8 minutes, from 0.5-2 minutes or from 1-5 minutes.
152. A biomass slurry/pretreated biomass/biomass preparation obtained or obtainable by the method of any one of embodiments 1 to 151.
153. A method for producing fermentable sugars, comprising subjecting the biomass slurry/pretreated biomass/biomass of embodiment 152 to a saccharification step.
154. The method of any one of embodiments 1 to 151, further comprising subjecting the resulting biomass slurry/pretreated biomass/biomass to a saccharification step to produce fermentable sugars.
155. The method of embodiment 153 or 154, wherein the saccharification step is carried out under conditions that yield 30% or more, 40% or more, 50% or more, 60% or more, or 70% or more of the theoretical yield of (i) glucose, (ii) xylose, (iii) cellobiose, (iv) both glucose and xylose, (v) both glucose and cellobiose, (vi) both xylose and cellobiose, or (vii) each of glucose, xylose and cellobiose in the biomass.
156. The method of any one of embodiments 153 to 155, further comprising culturing a fermenting microorganism in a medium comprising said fermentable sugars under conditions in which the fermenting microorganism produces fermentation product.
157. The method of embodiment 156, wherein the fermentation product is a fuel molecule.
158. The method of embodiment 156 or embodiment 157, in which the saccharification and fermentation are carried out separately.
159. The method of embodiment 156 or embodiment 157, in which the saccharification and fermentation are carried out simultaneously.
160. The method of any one of embodiments 157 to 159, wherein the fuel molecule is ethanol.
161. The method of any one of embodiments 157 to 160, further comprising recovering the fuel molecule.
162. The method of embodiment 161, wherein the recovery is by distillation.
163. The method of any method of any one of embodiments 1 to 162, which is carried out under conditions that result in at least 5%, at least 10%, at least 20%, at least 30%, at least 40%, or at least 50% greater product yield or product concentration as compared to a method in which biomass slurry not treated with said one or more hydrolyzing proteins.
164. The method of embodiment 163, wherein the product is a saccharification product.
165. The method of embodiment 163, wherein the product is a fermentation product.
166. The method of any one of embodiments 1 to 150 and 153 to 165, which is carried out under conditions that result in at least 5%, at least 10%, at least 20%, at least 30%, at least 40%, or at least 50% reduction in water usage in one or more steps as compared to a method in which biomass is not treated with said one or more hydrolyzing proteins.
167. The method of embodiment 166, wherein the reduction in water usage is in a pretreatment process, saccharification process, fermentation process, distillation process, or a combination of two, three or all four of the foregoing processes.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

TABLE 1

| Sample | 0 hrs. | 2 hrs. | 17 hrs. | 24 hrs. | 48 hrs. |
|---|---|---|---|---|---|
| Eucalyptus, 1x enzyme | 0% | 6.53% | 21.55% | 22.26% | 32.38% |
| Mixed Pine, 1x enzyme | 0% | 4.70% | 12.66% | 18.40% | 22.42% |
| Radiata Pine, 1x enzyme | 0% | 4.68% | 12.12% | 15.75% | 19.01% |
| Eucalyptus, 0.5x enzyme | 0% | 3.71% | 14.40% | 16.38% | 25.70% |
| Mixed Pine, 0.5x enzyme | 0% | 3.12% | 8.63% | 12.86% | 16.93% |
| Radiata Pine, 0.5x enzyme | 0% | 2.49% | 7.45% | 12.55% | 13.87% |
| Eucalyptus, 2x enzyme | 0% | 11.07% | 28.98% | 35.53% | 44.08% |
| Mixed Pine, 2x enzyme | 0% | 7.13% | 19.35% | 25.88% | 31.33% |
| Radiata Pine, 2x enzyme | 0% | 6.75% | 20.61% | 21.69% | 28.04% |
| Eucalyptus, no enzyme | 0% | | | | 0% |
| Mixed Pine, no enzyme | 0% | | | | 0% |
| Radiata Pine, no enzyme | 0% | | | | 0% |

TABLE 2

| | | | | | | 30 | Final (60 |
|---|---|---|---|---|---|---|---|
| | g/L | g/L | g/L | % Cellulose | % Hemicellulose | minute | minute) |
| Sample | cellobiose | glucose | xylose | Saccharification | Saccharification | cP | cP |
| 60° C. no enzyme control | 0.04 | 1.03 | 2.64 | 0.0% | 0.0% | 4165 | 5061 |
| 70° C. no enzyme control | 0.09 | 1.04 | 2.66 | 0.0% | 0.0% | 2851 | 3088 |
| 150 CTU 60° C. no buffer | 1.39 | 1.35 | 2.79 | 1.6% | 1.6% | 1473 | 1865 |
| 150 CTU 60° C. | 8.92 | 8.32 | 3.13 | 15.2% | 5.1% | 623 | 495 |
| 150 CTU 70° C. | 3.69 | 5.08 | 2.96 | 7.2% | 3.1% | 1434 | 1791 |
| 100 CTU 60° C. | 7.70 | 5.86 | 3.06 | 11.8% | 4.4% | 982 | 688 |
| 100 CTU 70° C. | 2.63 | 3.37 | 2.83 | 4.6% | 1.8% | 1239 | 1894 |
| 50 CTU 60° C. | 5.32 | 3.45 | 2.93 | 7.3% | 3.0% | 1142 | 1062 |
| 50 CTU 70° C. | 2.18 | 2.01 | 2.78 | 2.9% | 1.3% | 2133 | 2433 |
| 25 CTU 60° C. | 3.19 | 2.23 | 2.94 | 4.1% | 3.1% | 1731 | 1187 |
| 25 CTU 70° C. | 1.10 | 1.63 | 2.73 | 1.5% | 0.7% | 3403 | 3357 |

TABLE 3

| | | | Percent | Percent |
|---|---|---|---|---|
| Time | Visc | Temp | Residual | Viscosity |
| (min) | (cP) | (° C.) | Viscosity | Reduction |
| 0.0 | 5585 | 59.85 | 100.0% | 0.0% |
| 0.25 | 5004 | 60.20 | 89.6% | 10.4% |
| 0.5 | 4543 | 60.15 | 81.3% | 18.7% |
| 1 | 3321 | 60.05 | 59.5% | 40.5% |
| 2.0 | 2622 | 60.05 | 46.9% | 53.1% |
| 5.0 | 2583 | 60.05 | 46.2% | 53.8% |
| 10.0 | 1860 | 60.05 | 33.3% | 66.7% |
| 20.0 | 1766 | 60.05 | 31.6% | 68.4% |
| 29.3 | 1479 | 60.1 | 26.5% | 73.5% |
| 40.0 | 1259 | 60.05 | 22.5% | 77.5% |
| 60.0 | 1187 | 60.05 | 21.3% | 78.7% |

TABLE 4

| | | | Percent | Percent |
|---|---|---|---|---|
| Time | Visc | Temp | Residual | Viscosity |
| (min) | (cP) | (° C.) | Viscosity | Reduction |
| 0 | 7256 | 68.7 | 100.0% | 0.0% |
| 0.25 | 5932 | 70 | 81.8% | 18.2% |
| 0.5 | 5069 | 70.3 | 69.9% | 30.1% |
| 1 | 4509 | 70.2 | 62.1% | 37.9% |
| 2 | 3597 | 70.05 | 49.6% | 50.4% |
| 5 | 3250 | 70.05 | 44.8% | 55.2% |
| 10 | 3111 | 70.05 | 42.9% | 57.1% |
| 20 | 3783 | 70.0 | 52.1% | 47.9% |
| 30 | 3403 | 70.05 | 46.9% | 53.1% |
| 40 | 3641 | 70.05 | 50.2% | 49.8% |
| 60 | 3357 | 70.05 | 46.3% | 53.7% |

TABLE 5

| | | | | | | |
|---|---|---|---|---|---|---|
| RPM | Start | 60 | 300 | 600 | 900 | 1800 |

Initial viscosity (cp) at selected times (sec)

| | | | | | | |
|---|---|---|---|---|---|---|
| 100 | 4900 | 4789 | 4484 | 4199 | 3991 | 3652 |
| 50 | 1400 | 13484 | 12410 | 11434 | 10750 | 9701 |
| 30 | 27500 | 25592 | 22402 | 20348 | 19375 | 18590 |
| 20 | 35500 | 34061 | 31117 | 28317 | 26253 | 22783 |
| 10 | 68000 | 66697 | 61223 | 56415 | 53174 | 48524 |
| 6 | 120000 | 117304 | 106214 | 95859 | 88385 | 76284 |
| 5 | 173000 | 157785 | 118063 | 110504 | 109637 | 109525 |
| 3 | 300000 | 282657 | 218154 | 185800 | 174273 | 168182 |
| 2 | 300000 | 289247 | 246476 | 227931 | 222345 | 220003 |

Average steady-state viscosity (cp) at selected times (sec)

| | | | | | | |
|---|---|---|---|---|---|---|
| 100 | 3550 | 3500 | 3239 | 3102 | 3050 | 3021 |
| 50 | 7500 | 6857 | 6022 | 5933 | 5927 | 5927 |
| 30 | 12000 | 11408 | 10387 | 10126 | 10083 | 10075 |
| 20 | 13000 | 12774 | 12663 | 12527 | 12393 | 12011 |
| 10 | 24000 | 20971 | 20225 | 20223 | 20223 | 20223 |
| 6 | 40000 | 38418 | 30710 | 27855 | 27138 | 26902 |
| 5 | 51000 | 47319 | 37950 | 34666 | 33893 | 33659 |
| 3 | 100000 | 91533 | 69907 | 57845 | 52991 | 49935 |
| 2 | 118000 | 108301 | 79943 | 67647 | 63944 | 62391 |

TABLE 6

| | Hydrolysis | | Retention | Steam-Ex |
|---|---|---|---|---|
| Sample | Temp | | Time | Temp/ |
| Condition | (° C.) | % acid | (min) | pressure |
| A | 160 | 0.5% Sulfuric | 30 | 160° C./75 psig |
| B | 160 | 0.642% Nitric | 30 | 160° C./75 psig |
| C | 160 | 0.333% Phosphoric | 30 | 160° C./75 psig |
| D | 160 | 0.612% Acetic | 30 | 160° C./75 psig |
| E | 160 | No acid | 30 | 160° C./75 psig |
| | | (autohydrolysis) | | |

TABLE 7

| | | % | | |
|---|---|---|---|---|
| | | Insoluble | % | % |
| Sample | Condition | Solids | Glucan | Xylan |
| A1 | Sulfuric acid/unexploded | 30.8 | 66.5 | 3.5 |
| A2 | Sulfuric acid/steam-exploded | 26.9 | 61.7 | 2.7 |
| B1 | Nitric acid/unexploded | 31.6 | 63.0 | 1.4 |
| B2 | Nitric acid/steam-exploded | 27.1 | 64.7 | 1.0 |
| C1 | Phosphoric acid/unexploded | 32.2 | 58.3 | 8.1 |
| C2 | Phosphoric acid/steam-exploded | 27.4 | 58.2 | 7.5 |
| D1 | Acetic acid/unexploded | 31.6 | 50.6 | 16.4 |
| D2 | Acetic acid/steam-exploded | 28.1 | 52.9 | 16.0 |
| E1 | No acid/unexploded | 35.4 | 46.5 | 20.8 |
| E2 | No acid/steam-exploded | 31.3 | 48.0 | 20.1 |

TABLE 8

| Bauer McNett | | | | | | | | | | |
|---|---|---|---|---|---|---|---|---|---|---|
| Analysis | Sample ID | | | | | | | | | |
| % in size class | RA1 | RA2 | RB1 | RB2 | RC1 | RC2 | RD1 | RD2 | RE1 | RE2 |
| R15 | 46.7 | 44.8 | 28.3 | 30.5 | 55.3 | 58.9 | 67.4 | 66.0 | 72.9 | 69.5 |
| P15-R30 | 17.7 | 19.3 | 22.4 | 21.4 | 14.1 | 13.0 | 9.6 | 10.3 | 7.6 | 10.2 |
| P30-R50 | 12.2 | 11.9 | 17.0 | 14.2 | 10.6 | 9.6 | 8.4 | 8.2 | 6.8 | 7.1 |
| P50-R100 | 14.3 | 14.5 | 21.3 | 21.3 | 12.3 | 10.6 | 8.1 | 8.9 | 7.3 | 7.8 |
| P100-R200 | 9.1 | 9.6 | 11.0 | 12.6 | 7.7 | 7.8 | 6.5 | 6.6 | 5.4 | 5.4 |

TABLE 9

| | | A1 | A2 | B1 | B2 | C1 | C2 | D1 | D2 | E1 | E2 |
|---|---|---|---|---|---|---|---|---|---|---|---|
| FIBERS | Number of | 5021 | 5030 | 5039 | 5034 | 5015 | 5029 | 5032 | 5042 | 4662 | 3489 |
| | analysed fibers | | | | | | | | | | |
| | Mean fiber | 479 | 427 | 397 | 349 | 488 | 496 | 546 | 514 | 483 | 490 |
| | arithmetic | | | | | | | | | | |
| | length, microns | | | | | | | | | | |
| | Mean length- | 703 | 582 | 524 | 432 | 722 | 756 | 924 | 818 | 803 | 838 |
| | weighted fiber | | | | | | | | | | |
| | length, microns | | | | | | | | | | |
| | Mean fiber | 31.6 | 29.4 | 32.7 | 30.9 | 32.9 | 28.7 | 31.9 | 29.5 | 34.6 | 35.7 |
| | width, microns | | | | | | | | | | |
| | Fiber kink, % | | | | | | | | | | |
| | Average kink | 1.14 | 1.23 | 1.10 | 1.13 | 1.13 | 1.23 | 1.08 | 1.15 | 1.10 | 1.16 |
| | number | | | | | | | | | | |
| | Average kink | 128.11 | 125.58 | 126.74 | 123.43 | 121.56 | 127.41 | 120.08 | 124.00 | 113.37 | 123.54 |
| | angle, % | | | | | | | | | | |
| | Kinked fiber | 15.32 | 22.49 | 12.64 | 15.32 | 14.19 | 22.05 | 11.68 | 16.64 | 12.06 | 14.99 |
| | content, % | | | | | | | | | | |
| | Fiber curl index, | | | | | | | | | | |
| | % | | | | | | | | | | |
| | Mean fiber curl | 8.41 | 9.90 | 7.84 | 9.14 | 9.62 | 9.61 | 8.84 | 9.23 | 9.99 | 9.78 |
| | index | | | | | | | | | | |
| | Macro Fibrillation | 1.54 | 1.90 | 1.79 | 2.61 | 1.95 | 1.62 | 1.64 | 1.71 | 2.01 | 2.34 |
| | index, % | | | | | | | | | | |
| | Broken fiber | 46.14 | 47.97 | 49.94 | 53.90 | 51.81 | 44.68 | 48.21 | 46.90 | 51.15 | 53.32 |
| | content, % | | | | | | | | | | |
| FINES | Number of | 440412 | 600982 | 474132 | 1324238 | 466415 | 649426 | 588494 | 612548 | 605784 | 625919 |
| | analyzed fines | | | | | | | | | | |
| | Fine content, | 32.7 | 43.6 | 39.5 | 66.8 | 32.7 | 42.3 | 34.4 | 38.9 | 32.3 | 28.7 |
| | % in area | | | | | | | | | | |
| | Fine content, | 85.5 | 90.3 | 89.2 | 96.3 | 86.9 | 89.3 | 87.5 | 88.4 | 89.8 | 91.0 |
| | % in length | | | | | | | | | | |
| | Mean Fine area, | 999 | 925 | 997 | 854 | 1059 | 906 | 1032 | 1006 | 1018 | 913 |
| | microns² | | | | | | | | | | |
| | Mean fine length, | 36.5 | 36.5 | 37.0 | 36.5 | 37.7 | 35.0 | 37.0 | 37.0 | 37.0 | 34.8 |
| | microns | | | | | | | | | | |

TABLE 10

| | | | | | % | | | | | |
|---|---|---|---|---|---|---|---|---|---|---|
| | | | | | Reduction | | | | | |
| | | | Time Constant | | in Viscosity | Viscosity | | | Viscosity | |
| | Percent | | for Shear | Time Constant | (%) | Ratio | Viscosity | | at | Final |
| | Solids | RPM | Thinning | for Enzyme | (1 − | (%) | at | Viscosity at | 70 min. | Viscosity |
| Material | (%) | (1/min) | (sec) | (sec) | μend/μstart) | (μend/μstart) | start (cp) | −30 min (cp) | (cp) | (cp) |
| A1 | 10% | 20 rpm | 4.5 to 11.1 | 73 to 75 | 38.1% | 61.9% | 42000.0 | 26000.0 | | |
| A2 (no Enzyme) | 10% | 20 rpm | 2.1 to 12.1 | 22 to 252 | 10.8% | 89.2% | 18209.6 | 16250.0 | | |
| A2 | 10% | 20 rpm | 3.4 to 11.1 | 148 to 373 | 41.5% | 58.5% | 21500.0 | 12573.7 | | |
| B1 | 10% | 20 rpm | 1.9 to 8.6 | 153 to 183 | 85.5% | 14.5% | 20000.0 | 2900.1 | | |
| B2 (no Enzyme) | 10% | 20 rpm | 3.8 to 6.6 | 34 to 37 | 36.9% | 63.1% | 3000.0 | 1893.0 | | |
| B2 | 10% | 20 rpm | 2.1 to 5.9 | 66 to 143 | 35.0% | 65.0% | 2000.0 | 1300.0 | | |
| C1 | 5% | 20 rpm | 5.0 to 10.6 | 219 to 281 | 67.1 | 32.9% | 25000.0 | 8228.4 | | |
| C2 | 5% | 20 rpm | 4.7 to 10.7 | 201 to 219 | 56.2% | 43.8% | 11000.0 | 4820.8 | | |
| D1 | 5% | 20 rpm | 3.3 to 11.1 | 213 to 360 | 37.9% | 62.1% | 45000.0 | 27933.7 | | |
| D2 | 5% | 20 rpm | 3.6 to 13.7 | 199 to 264 | 72.7% | 27.3% | 30000.0 | 8202.5 | | |
| E1-95 min | 5% | 20 rpm | 4.0 to 10.4 | 1609 to 1625 | 38.8% | 51.2% | 45000.0 | 32000.0 | 28000.0 | 27545.4 |
| E2-110 min | 5% | 20 rpm | 1.4 to 8.3 | 2941 to 3612 | 56.0% | 44.0% | 41000.0 | 30000.0 | 21500.0 | 18026.0 |
| A2 (no Enzyme) | 10% | 3 rpm | 4.7 to 9.1 | 178 to 183 | 43.7% | 56.3% | 300000.0 | 169005.7 | | |
| A2 | 10% | 3 rpm | 2.7 to 8.3 | 206 to 334 | 59.1% | 40.9% | 220000.0 | 90021.1 | | |
| A2 (2X enzyme) | 10% | 3 rpm | 4.0 to 11.1 | 161 to 226 | 61.6% | 38.4% | 245000.0 | 94054.6 | | |
| A1 | 5% | 3 rpm | 3.3 to 10.7 | 687 to 870 | 65.5% | 34.5% | 80000.0 | 27592.8 | | |
| A2 | 5% | 3 rpm | 4.1 to 36.0 | 86 to 218 | 43.2% | 56.8% | 12500.0 | 7101.4 | | |
| B2 | 10% | 3 rpm | 1.5 to 12.9 | 179 to 219 | 53.1% | 46.9% | 17500.0 | 8200.4 | | |
| B2 (no Enzyme) | 10% | 3 rpm | 3.4 to 10.8 | 3.7 to 27 | 12.1% | 87.9% | 33000.0 | 29000.0 | | |
| B1 | 13% | 3 rpm | 4.0 to 14.2 | 181 to 183 | 31.0% | 69.0% | 29000.0 | 200004.3 | | |
| B2 | 13% | 3 rpm | 3.1 to 10.1 | 111 to 142 | 49.3% | 50.7% | 150000.0 | 76000.0 | | |

What is claimed is:

1. A method for producing biomass slurry, comprising mixing biomass which has been subject to steam explosion with an aqueous liquid in the presence of one or more hydrolyzing proteins under conditions that:
(a) are unfavorable for enzymatic saccharification by said one or more hydrolyzing proteins; and/or
(b) result in less than 40%, less than 30%, less than 20% or less than 10% glucan (e.g., glucose and/or cellobiose) saccharification by said one or more hydrolyzing proteins; and/or
(c) require at least 10%, at least 20%, at least 30%, or at least 40% less power to mix the biomass with the aqueous liquid as compared to mixing biomass and aqueous liquid in the absence of said hydrolyzing proteins over a 2-, 5-, 10-, 15- or 20-minute period; and/or
(d) permit mixing of a slurry containing at least 10%, at least 20%, at least 30%, or at least 40% more biomass solids without increasing power usage as compared to a slurry mixed in the absence of said hydrolyzing proteins,
thereby producing biomass slurry.

2. The method of claim 1, wherein the conditions yield 10% or less, 8% or less, 6% or less, 5% or less, 4% or less, 3% or less, or 2% or less of the theoretical yield of (i) glucose, (ii) xylose, (iii) cellobiose, (iv) both glucose and xylose, (v) both glucose and cellobiose, (vi) both xylose and cellobiose, or (vii) each of glucose, xylose and cellobiose in the biomass.

3. The method of claim 1, wherein the conditions are effective to reduce the viscosity of the biomass by at least 10%, by at least 20%, by at least 30%, by at least 40% or by at least 50%.

4. The method of claim 1, wherein the steam explosion has been carried out under conditions that reduce the viscosity of the biomass by at least 10%, by at least 20%, by at least 30%, by at least 40% or by at least 50%.

5. The method of claim 1, wherein the one or more hydrolyzing proteins are at a dose of 5 μg to 40 mg, 5 μg to 30 mg or 5 n to 20 mg protein or 10-250 CTUs per gram dry weight of biomass.

6. The method of claim 1, wherein the biomass has been subject to acid pretreatment.

7. The method of claim 1, wherein the mixing is carried out at a temperature of 50° C. to 100° C., 60° C. to 100° C., or 50° C. to 100° C., 60° C. to 100° C., or 50° C. to 80° C.

8. The method of claim 7, wherein the mixing is carried out at a temperature of 65° C. to 75° C., 62° C. to 72° C., or 62° C. to 75° C.

9. The method of claim 1, wherein the mixing is carried out for a period of at least 0.25 minutes, at least 0.5 minute, at least 1 minute or at least 2 minutes, at least 5 minutes, at least 10 minutes, or at least 15 minutes.

10. The method of claim 1, wherein the mixing is carried out for a period of up to 30 minutes, up to 1 hour or up to 1.5 hours.

11. The method of claim 1, wherein the biomass and the aqueous liquid are at a 1:1 to 1:7, 1:2 to 1:6, 1:1 to 1:7, 1:2 to 1:6, 1:2.5 to 1:5.7, 1:3.33 to 1:5.7, or 1:4 to 1:5.7 solid:liquid weight ratio.

12. A method for continuous production of biomass slurry, comprising:
(a) combining biomass which has been subject to steam explosion, an aqueous liquid and one or more hydrolyzing proteins into a vessel comprising a biomass slurry, wherein the biomass, an aqueous liquid and one or more hydrolyzing proteins are introduced into the vessel at a rate in which the slurry viscosity in the vessel is maintained,
(b) simultaneously pumping slurry out of the vessel at a rate that maintains the slurry volume in the vessel;
wherein the conditions in the vessel:
(i) are unfavorable for enzymatic saccharification by said one or more hydrolyzing proteins; and/or
(ii) result in less than 40%, less than 30%, less than 20% or less than 10% glucan (e.g., glucose and/or cellobiose) saccharification by said one or more hydrolyzing proteins; and/or
(iii) require at least 10%, at least 20%, at least 30%, or at least 40% less power to mix the biomass with the aqueous liquid as compared to mixing biomass and aqueous liquid in the absence of said hydrolyzing proteins over the residence time of the biomass in the vessel; and/or
(iv) permit mixing of a slurry containing at least 10%, at least 20%, at least 30%, or at least 40% more biomass solids without increasing power usage as compared to a slurry mixed in the absence of said hydrolyzing proteins,
thereby continuously producing biomass slurry.

13. The method of claim 12, wherein the steam explosion has been carried out under conditions that reduce the viscosity of the biomass by at least 10%, by at least 20%, by at least 30%, by at least 40% or by at least 50%.

14. The method of claim 12, wherein the one or more hydrolyzing proteins are at a dose of 5 μg to 40 mg, 5 μg to 30 mg or 5 μg to 20 mg protein or 10-250 CTUs per gram dry weight of biomass.

15. The method of claim 12, wherein the biomass has been subject to acid pretreatment.

16. The method of claim 12, wherein the vessel is maintained at a temperature of 50° C. to 100° C., 60° C. to 100° C., or 50° C. to 80° C.

17. The method of claim 16, wherein the vessel is maintained at a temperature of 65° C. to 75° C., 62° C. to 72° C., or 62° C. to 75° C.

18. The method of claim 12, wherein the biomass slurry comprises at least 5%, at least 8% or at least 10% weight solids pretreated with one or more hydrolyzing proteins in a dose of 5 μg to 40 mg, 5 μg to 30 mg or 5 μg to 20 mg protein or 10-250 CTUs per gram dry weight of biomass.

19. The method of claim 18, wherein the solids are from biomass which has been subject to steam explosion prior to said pretreatment with hydrolyzing proteins.

20. The method of claim 19, wherein the steam explosion prior to pretreatment has been carried out under conditions that reduce the viscosity of the biomass by at least 10%, by at least 20%, by at least 30%, by at least 40% or by at least 50%.

21. The method of claim 12, wherein the vessel is a continuous stirred tank reactor (“CSTR”).

22. The method of claim 12, wherein the vessel is a plug flow reactor (“PFR”).

23. The method of claim 12, which comprises continuously producing biomass slurry for a period of time of at least 12 hours or at least 18 hours.

24. The method of claim 23, which comprises continuously producing biomass slurry for a period of time of up to 24 hours, up to 36 hours, up to 48 hours, up to 72 hours, up to 96 hours, up to 1 week, up to 2 weeks, up to 3 weeks, up to 1 month, up to 6 months, or up to 1 year.

25. The method of claim 23, in which the vessel is not cleaned during said period of time.

26. The method of claim 12, in which 3% to 10% of the slurry volume is pumped out of the vessel every minute.

27. The method of claim 12, in which the slurry has a residence time of less than 2 hours in the vessel.

28. The method of claim 27, wherein the slurry has a residence time of 2 minutes to 30 minutes in the vessel.

29. The method of claim 12, further comprising, prior to step (a), forming said biomass slurry.

30. The method of claim 29, wherein forming said biomass slurry comprises combining in said vessel biomass which has been subject to steam explosion with an aqueous liquid in the presence of one or more hydrolyzing proteins.

31. The method of claim 30, wherein the steam explosion has been carried out under conditions that reduce the viscosity of the biomass by at least 10%, by at least 20%, by at least 30%, by at least 40% or by at least 50%.

32. The method of claim 30, wherein said one or more hydrolyzing proteins are at a dose of 5 μg to 40 mg, 5 μg to 30 mg or 5 μg to 20 mg protein or 10-250 CTUs per gram dry weight of biomass.

33. The method of claim 30, wherein the biomass and the aqueous liquid are combined at a 1:1 to 1:7, 1:2 to 1:6, 1:2.5 to 1:5.7, 1:3.33 to 1:5.7, or 1:4 to 1:5.7 solid:liquid weight ratio.

34. The method of claim 30, wherein the vessel is maintained at a temperature of 50° C. to 100° C., 60° C. to 100° C., or 50° C. to 80° C.

35. The method of claim 35, wherein the vessel is maintained at a temperature in the range of 65° C. to 75° C., 62° C. to 72° C., or 62° C. to 75° C.

36. The method of claim 30, which further comprises agitating the vessel contents during slurry formation.

37. The method of claim 36, wherein the vessel contents are agitated for a period of at least 0.5 minute, at least 1 minute or at least 2 minutes, at least 5 minutes, at least 10 minutes, or at least 15 minutes.

38. The method of claim 36, wherein the vessel contents are agitated for a period of up to 1 hour or up to 1.5 hours.

39. The method of claim 30, wherein the biomass has been subject to acid pretreatment.
